(19) United States
(12) Patent Application Publication
(10) Pub. No.: US 2013/0203969 A1
(43) Pub. Date: Aug. 8, 2013

(54) USE OF SMALL MOLECULES IN METHODS FOR PURIFICATION OF BIOMOLECULES

(71) Applicants: Jad Jaber, Sudbury, MA (US); Wilson Moya, Concord, MA (US); Ajish Potty, Woburn, MA (US); Alison Dupont, Sandown, NH (US); Matthew T. Stone, Cambridge, MA (US); Mikhail Kozlov, Lexington, MA (US)

(72) Inventors: Jad Jaber, Sudbury, MA (US); Wilson Moya, Concord, MA (US); Ajish Potty, Woburn, MA (US); Alison Dupont, Sandown, NH (US); Matthew T. Stone, Cambridge, MA (US); Mikhail Kozlov, Lexington, MA (US)

(73) Assignee: EMD Millipore Corporation, Billerica, MA (US)

(21) Appl. No.: 13/566,320

(22) Filed: Aug. 3, 2012

Related U.S. Application Data
(60) Provisional application No. 61/666,287, filed on Jun. 29, 2012.
(60) Provisional application No. 61/575,376, filed on Aug. 19, 2011.

(51) Int. Cl. C07K 16/00

(52) U.S. Cl. CPC C07K 16/00
USPC 530/388.1

(57) ABSTRACT

The present invention relates to novel and improved methods for the purification of biomolecules. In particular, the present invention relates to methods of protein purification which employ small molecules, which include at least one non-polar group and at least one cationic group or which include at least one non-polar group and at least one anionic group.

### RELATED APPLICATIONS

The present patent application claims the benefit of priority of U.S. Provisional Patent Application Nos. 61/575,376, filing date Aug. 19, 2011, and U.S. Provisional Patent Application No. 61/666,287, filing date Jun. 29, 2012, the entire contents of each of which are incorporated by reference herein.

### FIELD

The present invention relates to novel and improved methods for purification of biomolecules. In particular, the present invention relates to methods of protein purification which employ small molecules.

### BACKGROUND

The general process for the manufacture of biomolecules, such as proteins and particularly recombinant therapeutic proteins, typically involves two main steps: (1) the expression of the protein in a host cell, and (2) the purification of the protein. The first step generally involves growing the desired host cells in a bioreactor to facilitate the expression of the protein of interest. Once the protein is expressed at the desired levels, the protein is removed from the host cells and harvested. Suspended materials, such as cells, cell fragments, lipids and other insoluble matter are typically removed from the protein-containing fluid by filtration or centrifugation, resulting in a clarified fluid containing the protein of interest in solution along with various soluble impurities.

The second step generally involves the purification of the harvested protein to remove the soluble impurities. Examples of soluble impurities include host cell proteins (generally referred to as HCPs, which are cellular proteins other than the desired or targeted protein), nucleic acids, endotoxins, viruses, protein variants and protein aggregates.

This purification typically involves several chromatography steps, which may include one or more of bind and elute hydrophobic interaction chromatography (HIC); flow-through hydrophobic interaction chromatography (FTHIC); mixed mode chromatography techniques, e.g., bind and elute weak cation and anion exchange, bind and elute hydrophobic and ion exchange interaction and flow-through hydrophobic and ion exchange mixed mode interaction (FTMM), both of which can utilize resins such as Capto Adhere, Capto MMC, HEA Hypercel, PPA Hypercel.

Other alternative methods for purifying proteins have been investigated in recent years, one such method involves a flocculation technique. In this technique, a soluble polyelectrolyte is added to an unclarified cell culture broth to capture the suspended materials and a portion of the soluble impurities thereby forming a flocculant, which is subsequently removed from the protein solution by filtration or centrifugation.

Alternatively, a soluble polyelectrolyte may be added to clarified cell culture broth to capture the protein of interest, thereby forming a flocculant, which is allowed to settle and can be subsequently isolated from the rest of the solution. The flocculant is typically washed to remove loosely adhering impurities. Afterwards, an increase in the solution's ionic strength brings about the dissociation of the target protein from the polyelectrolyte, subsequently resulting in the resolubilization of the polyelectrolyte into the protein-containing solution.

The main drawback of this flocculation technique is that it requires the use of polymers that may end up with the target protein, may be toxic and/or not easily cleared from the patient's body, are potentially expensive in terms of single use applications, and not readily available as they often need to be synthesized.

### SUMMARY OF THE INVENTION

The present invention provides improved processes for purification of biomolecules, where the processes employ materials that are less toxic, are easy to handle and are readily available. Further, in some embodiments, the processes according to the claimed invention obviate the need to use expensive reagents and chromatography steps, e.g., Protein A affinity chromatography.

The present invention relates to methods of using certain small molecules which are capable of binding to a biomolecule of interest such as a target molecule, e.g., a monoclonal antibody (the process referred to as “capture”), as well as small molecules which bind to a soluble or an insoluble impurity, e.g., host cell proteins, DNA, virus, whole cells, cellular debris, endotoxins etc., in a biological material containing stream, in order to purify the target protein or separate the target protein from the impurity. In some embodiments, methods described herein are particularly useful in the removal of insoluble impurities from a sample containing a protein of interest (the process referred to as “clarification”).

In some embodiments, the present invention relates to a method of separating a target biomolecule from one or more insoluble impurities in a sample; the method comprising the steps of: (i) providing a sample comprising a target biomolecule and one or more insoluble impurities; (ii) contacting the sample with a small molecule comprising at least one cationic group and at least one non-polar group, in an amount sufficient to form a precipitate comprising the one or more insoluble impurities; and (iii) removing the precipitate from the sample, thereby to separate the target molecule from the one or more insoluble impurities.

In some embodiments, the present invention relates to a method of purifying an antibody in a sample; the method comprising the steps of: (i) providing a sample comprising an antibody and one or more insoluble impurities; (ii) contacting the sample with a small molecule comprising at least one cationic group and at least one non-polar group, in an amount sufficient to form a precipitate comprising the one or more insoluble impurities and a liquid phase comprising the antibody; and (iii) subjecting the liquid phase to at least one chromatography step, thereby to purify the target antibody.

In some embodiments, the at least one chromatography step is an affinity chromatography step. In a particular embodiment, the affinity chromatography step comprises the use of a Protein A based affinity ligand.

In some embodiments, the small molecule comprises a non-polar group which is aromatic. In other embodiments, the small molecule comprises a non-polar group which is aliphatic.

In some embodiments, the one or more insoluble impurities are cells. In some embodiments, a small molecule comprising at least one cationic group and at least one non-polar group is selected form the group consisting of a monoalkyltrimethyl ammonium salt (non-limiting examples include cetyltrimethylammonium bromide or chloride, tetradecyltrimethylammonium bromide or chloride, alkyltrimethyl ammonium chloride, alkylaryltrimethyl ammonium chloride, dodecyltrimethylammonium bromide or chloride, dodecyldimethyl-2-phenoxyethylammonium bromide, hexadecylamine chloride or bromide, dodecyl amine or chloride, and cetyldimethylethyl ammonium bromide or chloride), a monoalkyldimethylbenzyl ammonium salt (non-limiting examples include alkyldimethylbenzyl ammonium chloride and benzethonium chloride), a dialkyldimethyl ammonium salt (non-limiting examples include domiphen bromide, didecyldimethyl ammonium chloride or bromide and octyldodecyldimethyl ammonium chloride or bromide), a heteroaromatic ammonium salt (non-limiting examples include cetylpyridium halides (chloride or bromide salts) and hexadecylpyridinium bromide or chloride, cis-isomer 1-[3-chloroallyl]-3,5,7-triaza-1-azoniaadamantane, alkyl-isoquinolinium bromide, and alkyldimethylnaphthylmethyl ammonium chloride), a polysubstituted quaternary ammonium salt, (non-limiting examples include alkyldimethylbenzyl ammonium saccharinate and alkyldimethylethylbenzyl ammonium cyclohexylsulfamate), and a bis-quaternary ammonium salt (non-limiting examples include 1,10-bis(2-methyl-4-aminoquinolinium chloride)-decane, 1,6-Bis{1-methyl-3-(2,2,6-trimethyl cyclohexyl)-propyldimethyl ammonium chloride]hexane or triclobisonium chloride, and the bis-quat referred to as CDQ by Buckman Brochures).

In a particular embodiment, the small molecule is benzethonium chloride

In some embodiments of the methods according to the present invention, 0.01 to 2.0% wt/vol of a small molecule is added to a sample to precipitate the one or more insoluble impurities. In some embodiments, such small molecules are employed during a clarification process step used in a protein purification process. In some embodiments, such a process is a continuous process.

In some embodiments, one or more small molecules described herein are used during a clarification step of a protein purification process, where such small molecules may be added directly to a bioreactor containing a cell culture, in order to precipitate one or more impurities. In other embodiments, one or more small molecules described herein may be employed during one or more other process steps in a purification process, e.g., as described in the Examples herein.

In some embodiments, the amount of a small molecule that is added is in solution form having a concentration ranging from 1 to 200 mg/ml.

In some embodiments, the precipitation step is carried out at a pH ranging from 2 to 9.

In some embodiments, the precipitate is removed from the sample by filtration (e.g., depth filtration). In other embodiments, the precipitate is removed from the sample by centrifugation.

In some embodiments, methods of separating a target biomolecule from one or more insoluble impurities further comprises the step of removing residual amounts of small molecule from the sample. In some methods, such a step comprises contacting the recovered solution with a polyanion or an adsorbent material to remove residual amounts of small molecules. In a particular embodiment, such a step employs activated carbon to remove the residual amounts of small molecule.

Also encompassed by the present invention are methods of purifying a target biomolecule from a sample comprising the target molecule along with one or more soluble impurities, where the method comprises the steps of: (i) contacting the sample with a small molecule comprising at least one anionic group and at least one non-polar group, in an amount sufficient to form a precipitate comprising the target molecule; and (ii) recovering the precipitate, thereby to separate the target biomolecule from the one or more soluble impurities.

In some embodiments, the small molecule comprises a non-polar group which is aromatic. In other embodiments, the small molecule comprises a non-polar group which is aliphatic.

In some embodiments, the sample is subjected to a clarification step prior to contacting it with the small molecule comprising at least one anionic group and at least one non-polar group. Exemplary clarification techniques include, but are not limited to, filtration and centrifugation.

In some embodiments, clarification is achieved by subjecting the sample to a small molecule comprising at least one cationic group and at least one non-polar group, as discussed above.

Exemplary small molecules comprising at least one anionic group and at least one non-polar group include, but are not limited to pharmaceutically relevant compounds such as, pterin derivatives (for example folic acid, pteroic acid), etacrynic acid, fenofibric acid, mefenamic acid, mycophenolic acid, tranexamic acid, zoledronic acid, acetylsalicylic acid, arsanilic acid, ceftiofur acid, meclofenamic acid, ibuprofine, naproxen, fusidic acid, nalidixic acid, chenodeoxycholic acid, ursodeoxycholic acid, tiaprofenic acid, niflumic acid, trans-2-hydroxycinnamic acid. 3-phenylpropionic acid, probenecid, clorazepate, icosapent, 4-acetamidobenzoic acid, ketoprofen, tretinoin, adenylosuccinic acid, naphthalene-2,6-disulfonic acid, tamibarotene, etodolacetodolic acid and benzylpenicillinic acid (see, e.g., DrugBank 3.0: a comprehensive resource for research on drugs. Knox C. Law V, Jewison T. Liu P, Ly S, Frolkis A, Pon A, Banco K, Mak C, Neveu V, Djoumbou Y, Eisner R, Guo A C. Wishart D S. Nucleic Acids Res. 2011 January; 39(Database issue):D1035-41. PMID: 21059682).

In a particular embodiment, a small molecule comprising at least one anionic group and at least one non-polar group (e.g., an aromatic group) is folic acid or a derivative thereof.

In some embodiments, a small molecule comprising at least one anionic group and at least one non-polar group is a dye molecule. Exemplary dyes include, but are not limited to, Amaranth and Nitro red.

In some embodiments, a small molecule is added to a concentration ranging from 0.001% to 5.0%.

In some embodiments, the pH of the sample is adjusted prior to the addition of the small molecule.

In some embodiments, the precipitation step is carried out at a pH ranging from 2 to 9.

In some embodiments, at least 50%, or at least 60%, or at least 70%, or at least 80%, or at least 90% or greater than 90% of the initial target biomolecule amount (e.g., target protein) present in the sample, is precipitated using the methods according to the present invention.

In some embodiments, less than 50%, or less than 40%, or less than 30%, or less than 20%, or less than 15%, or less than 10%, or less than 5% of the initial impurity level remains in the precipitate comprising the target biomolecule of interest following precipitation using a small molecule, as described herein. However, in some instances, a greater impurity level may precipitate with the target biomolecule.

In some embodiments, following the precipitation of a target biomolecule using a small molecule, as described herein, the precipitate is dissolved in a buffer having a pH ranging from 4.5 to 10.

In some embodiments, one or more static mixers are used for adding one or more small molecules to a sample.

In some embodiments, following the precipitation of the target biomolecule, the target biomolecule is subjected to a further chromatography step selected from the group consisting of ion exchange chromatography, hydrophobic interaction chromatography, affinity chromatography and mixed mode chromatography.

Exemplary target biomolecules include, but are not limited to, recombinant proteins, monoclonal antibodies and functional fragments, humanized antibodies, chimeric antibodies, polyclonal antibodies, multispecific antibodies, immunoadhesin molecules and CH2/CH3 region-containing proteins. The target biomolecule may be expressed in a mammalian expression system (e.g., CHO cells) or a non-mammalian expression system (e.g., bacterial, yeast or insect cells). The methods described herein maybe used in the context of proteins expressed using mammalian expression systems as well as non-mammalian expression systems.

### BRIEF DESCRIPTION OF THE DRAWINGS

FIG. 1 depicts a calibration curve for quantifying amounts of BZC in solution. The calibration curve was derived from a turbidimetric assay where known amounts of BZC and sodium tetrafloroborate are mixed to form a precipitate. The x-axis refers to the starting concentration of BZC in solution (ppm) and the y-axis refers to the turbidity (NTU) generated in solution upon the addition of a known amount of tetrafloroborate. The limit of detection of this assay is 100 mg/L or 100 ppm BZC in solution.

FIG. 2 depicts a graph demonstrating the results of a static binding experiment used to determine the capacity of activated carbon to bind BZC in solution. The x-axis refers to the mass of activated carbon added (g) and y-axis refers to the concentration of BZC remaining in solution after 10 minutes of mixing with activated carbon (mg/L). As demonstrated, 0.1 g of activated carbon is enough to reduce the starting amount of BZC in solution (25 mg) to an undetected level (i.e., less than 100 mg/L).

FIG. 3 depicts a graph representing the results of an optimization study where the optimal concentration of BZC to achieve maximum recovery of a target biomolecule (e.g., a monoclonal antibody MAb molecule) as well as maximum impurity clearance was found to be 4 g/L. The x-axis refers to the concentration of BZC (mg/ml) added to the feed to be clarified. The y-axis refers to the percentage (%) of HCP removed from the feed as a result of the clarification process with BZC (bars). The secondary y-axis refers to the percentage (%) of MAb that remained in the feed after the clarification process (depicted by diamonds).

FIG. 4 depicts a graph representing the results of an experiment to investigate the effect of solution pH on the precipitation efficiency of MAb by folic acid. More basic solution pH results in higher mass ratio of folic acid to MAb required to precipitate 90% or more of the MAb in solution. The x-axis refers to the mass ratio of folic acid to MAb added to the feed (mg/mg). The y-axis refers to the percentage (%) of MAb remaining in solution after precipitation with folic acid. Diamonds, squares, triangles and circles refer to the binding at pH of 4.5, 5.0, 5.5 and 6.0, respectively. Dotted lines are included as a guide.

FIG. 5 depicts a calibration curve for quantifying amounts of folic acid in solution. The calibration curve was derived from absorbance measurements at 350 nm of folic acid solutions of known concentration. The x-axis refers to the starting concentration of folic acid in solution (mg/ml) and the y-axis refers to the absorbance (arbitrary units) of the folic acid solutions at 350 nm. The limit of detection of this assay is 10 mg/L or 10 ppm folic acid in solution.

FIG. 6 depicts a graph representing the results of a binding isotherm experiment used to determine the capacity of activated carbon to bind folic acid in solution. The x-axis refers to the concentration of folic acid left in solution (mg/ml) after 10 minutes of mixing with activated carbon and the y-axis refers to the mass of folic acid (mg) bound per mass of activated carbon added (g) after 10 min of mixing. One gram of activated carbon is sufficient to remove 225 mg folic acid.

FIG. 7 depicts a graph representing the results of an experiment to investigate the MAb precipitation efficiency by Nitro red dye at a binding pH of 4.5. Nitro red/MAb ratio of at least 0.8 is required for complete precipitation of MAb. The x-axis refers to the mass ratio of folic acid to MAb added to the feed (mg/mg). The y-axis refers to the fraction of MAb remaining in solution after precipitation with Nitro red.

FIG. 8 depicts a graph demonstrating the effect of the binding pH on elution recovery for Nitro red precipitated MAb. Binding at a higher pH resulted in better elution recovery. The x-axis describes the sample and solution conditions tested. MAb is referred to as “Mab04”; supernatant is referred to as “Sup”; eluant is referred to as “Elu” and the numbers, 3.9, 4.45, and 4.9, refer to the solution pH where Nitro red bound and precipitated the MAb. The y-axis refers to the percentage (%) of MAb remaining in solution after precipitation (i.e., in the Sup) or after elution (i.e., in the Elu).

FIG. 9 depicts weak-cation exchange chromatograms used to evaluate charge variants in feed (trace labeled as Pure IgG) and elution samples form Amaranth dye molecule treated feeds (traces labeled as Amaranth elution 1 and 2). The x-axis refers to time (in minutes) and the y-axis refers to the absorbance of the feed and elution samples at 280 nm. Amaranth 1 and 2 are elution samples from duplicate experiments. This experiment is intended to show the reproducibility of the precipitation process using the Amaranth dye molecule.

FIG. 10 depicts a graph demonstrating the effect of shear on mean particle size of precipitate formed using folic acid. The x-axis refers to Shear rate (Sec⁻¹) generated by varying the flow rate inside a hollow fiber device and the y-axis refers to the mean particle size (micro meter) of the precipitate, as measured by a Malvern instrument. Triangle, square and diamond symbols refer to the solution pHs of 4, 5 and 5.5, respectively, where Nitro red bound and precipitated the MAb. Particles appear to be more compact and more resistant to shear at a lower pH.

FIG. 11a illustrates the set-up used to measure Flux vs. TMP for hollow fiber TFF system operating in complete recycle mode. Feed used was generated by mixing folic acid and clarified feed at pH 4.5 and 1:1 mass ratio to form a precipitate. A pump was used to deliver the precipitate to the hollow fiber device.

FIG. 11b depicts Flux versus TMP curves for folic acid-MAb precipitate using a 0.2 μm membrane at 3 different shear rates and 0.85 g/L MAb concentration. This experiment was carried out to determine the optimal conditions for operating the TFF system. The x-axis refers to the flux used (LMH)) and the y-axis refers to the measured Trans membrane pressure (Psi). Closed triangle, diamond and square symbols refer to shear rates of 850, 1700 and 3400 Sec⁻¹, respectively. The open symbol indicates that the system is at steady state until that point, beyond which an increase in TMP was observed with time indicating membrane fouling. It could be inferred from the Flux vs. TMP curves that the optimal shear and flow rates rate are 1700 S⁻¹ and 190 LMH respectively.

FIG. 11c depicts a graph representing single-pass concentration factor versus flux for folic acid-MAb precipitate using a 0.2 μm membrane at 3 different shear rates and 0.85 g/L MAb concentration. This experiment was carried out to determine the maximum concentration factor that can be achieved under optimal operating conditions. The x-axis refers to the flux used (LMH) and the y-axis refers to the concentration factor. Closed triangle, diamond and square symbols refer to the shear rates of 850, 1700 and 3400 Sec⁻¹, respectively. The open symbol indicates that the system is at steady state until that point, beyond which an increase in TMP was observed with time indicating membrane fouling. It could be inferred from the Flux vs. CF curves that under the respective optimal shear and flow rates rate of 1700 S⁻¹ and 190 LMH, respectively, the maximum concentration factor is 2.5×.

FIG. 12a depicts a graph representing Flux versus TMP curves for folic acid-MAb precipitate using a 0.2 μm membrane at 3 different shear rates and 4.3 g/L MAb concentration. This experiment was carried out to determine the optimal conditions for operating the TIT system with higher starting volumes of precipitate. The x-axis refers to the flux used (LMH) and the y-axis refers to the measured Trans membrane pressure (Psi). Closed triangle, diamond and square symbols refer to the shear rates of 850, 1700 and 3400 Sec⁻¹, respectively. The open symbol indicates that the system is at a steady state until that point, beyond which an increase in TMP was observed with time indicating membrane fouling. It could be inferred from the Flux vs. TMP curves that the optimal shear and flow rates rate are 1700 S⁻¹ and 170 LM respectively.

FIG. 12b a graph representing single-pass concentration factor versus flux for folic acid-MAb precipitate using a 0.2 μm membrane at 3 different shear rates and 4.3 g/L MAb concentration. This experiment was carried out to determine the maximum concentration factor that can be achieved under optimal operating conditions. The x-axis refers to the flux used (LMH) and the y-axis refers to the concentration factor. Closed triangle, diamond and square symbols refer to the shear rates of 850, 1700 and 3400 Sec⁻¹, respectively. The open symbol indicates that the system is at a steady state until that point, beyond which an increase in TMP was observed with time indicating membrane fouling. It could be inferred from the Flux vs. CF curves that under the optimal shear and flow rates rate of 1700 S⁻¹ and 170 LMH respectively, the maximum concentration factor is 2.2×.

FIG. 13 illustrates the set-up used for continuous concentration and washing of solids using hollow fiber modules. The binding step comprises two stages (i.e. two hollow fiber modules) where the precipitate is concentrated up to ˜4× and the wash step comprises three stages (i.e. three hollow fiber modules) where the concentrated precipitate is washed in a counter-current mode.

### DETAILED DESCRIPTION

The present invention is based, at least in part, on the discovery of use of certain types of small molecules in processes for purifying a biomolecule of interest, where the processes eliminate one or more steps, thereby reducing the overall operational cost and time.

Further, the present invention provides methods which employ small molecules that are readily available and are less toxic, should they end up with the therapeutic molecule, relative to other reagents that are used in a similar fashion in the art. Additionally, the small molecules used in the methods described herein enable processing of high density feed stock and are potentially disposable.

In order that the present disclosure may be more readily understood, certain terms are first defined. Additional definitions are set forth throughout the detailed description.

### I. DEFINITIONS

The term “small molecule,” as used herein, refers to a low molecular weight compound, which is not a polymer. The term encompasses molecules having a molecular weight of less than about 10,000 Daltons or less than about 9000 Daltons or less than about 8000 Daltons or less than about 7000 Daltons or less than about 6000 Daltons or less than about 5000 Daltons or less than about 4000 Daltons or less than about 3000 Daltons or less than about 2000 Daltons or less than about 1000 Daltons or less than about 900 Daltons or less than about 800 Daltons. Small molecules include, but are not limited to, organic, inorganic, synthetic or natural compounds. In various embodiments described herein, small molecules are used for the precipitation of either one or more impurities (i.e., clarification) or for the precipitation of a target biomolecule (i.e., capture). In some embodiments, the small molecules used in the methods according to the claimed invention are used for binding and precipitating an impurity (e.g., an insoluble impurity). Such small molecules are generally non-polar and cationic. In some other embodiments, the small molecules used in the methods according to the claimed invention are used for binding and precipitating a target biomolecule (e.g., a protein product). Such small molecules are generally non-polar and anionic.

The term “hydrophobic” or “non-polar,” as used interchangeably herein, refers to a compound or a chemical group or entity, which has little to no affinity for water. In some embodiments, the present invention employs small molecules that are non-polar or hydrophobic in nature. In some embodiments, a non-polar chemical group or entity is aromatic. In some other embodiments, a non-polar chemical group or entity is aliphatic.

The term “anionic” as used herein, refers to a compound or a chemical group or entity that contains a net negative charge.

The term “cationic” as used herein, refers to a compound or a chemical group or entity that contains a net positive charge.

The term “aromatic” as used herein, refers to a compound or a chemical group or entity in a molecule, in which at least a portion of the molecule contains a conjugated system of single and multiple bonds.

The term “aliphatic,” as used herein, refers to a compound or a chemical group or entity in a molecule, in which at least a portion of the molecule contains a acyclic or cyclic non-aromatic structure.

The term “target biomolecule,” “target protein,” “desired product,” “protein of interest,” or “product of interest,” as used interchangeably herein, generally refer to a polypeptide or product of interest, which is desired to be purified or separated from one or more undesirable entities, e.g., one or more soluble and/or insoluble impurities, which may be present in a sample containing the polypeptide or product of interest. The terms “target biomolecule,” “protein of interest,” “desired product” and “target protein,” as used interchangeably herein, generally refer to a therapeutic protein or polypeptide, including but not limited to, an antibody that is to be purified using the methods described herein.

As used herein interchangeably, the term “polypeptide” or “protein,” generally refers to peptides and proteins having more than about ten amino acids. In some embodiments, a small molecule, as described herein, is used to separate a protein or polypeptide from one or more undesirable entities present in a sample along with the protein or polypeptide. In some embodiments, the one or more entities are one or more impurities which may be present in a sample along with the protein or polypeptide being purified. As discussed, above, in some embodiments according to the methods described herein, a small molecule comprising at least one non-polar group and at least one anionic group is used for precipitating one or more impurities (e.g., insoluble impurities) in a sample comprising a target biomolecule. In some embodiment, insoluble impurities are whole cells.

In other embodiments according to the methods described herein, a small molecule comprising at least one cationic group and at least one non-polar group is used for precipitating a target biomolecule from a sample comprising the target biomolecule and one or more impurities (e.g. soluble impurities). Examples of impurities (soluble and insoluble) include e.g., host cell proteins, endotoxins. DNA, viruses, whole cells, cellular debris and cell culture additives etc.

In some embodiments, a protein or polypeptide being purified using the methods described herein is a mammalian protein, e.g., a therapeutic protein or a protein which may be used in therapy. Exemplary proteins include, but are not limited to, for example, renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; Dnase; IgE; a cytotoxic T-lymphocyte associated antigen (CTLA), such as CTLA-4; inhibin; activin; vascular endothelial growth factor (VEGF); receptors for hormones or growth factors; Protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as α-FGF and β-FGF; epidermal growth factor (EGF); transforming growth factor (TGF) such as TGF-alpha and TGF-beta, including TGF-β1, TGF-β2, TGF-β3, TGFβ4, or TGF-β5; insulin-like growth factor-I and -II (IGF-I and IGF-II); des(1-3)-IGF-I (brain IGF-I), insulin-like growth factor binding proteins (IGFBPs); CD proteins such as CD3, CD4, CD8, CD19 CD20, CD34, and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (Ils), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulator proteins; integrins such as CD11a, CD11b, CD11c, CD18, an ICAM, VLA-4 and VCAM; a tumor associated antigen such as HER2, HER3 or HER4 receptor; and fragments and/or variants of any of the above-listed polypeptides.

Further, in some embodiments, a protein or polypeptide purified using the methods described herein is an antibody, functional fragment or variant thereof. In some embodiments, a protein of interest is a recombinant protein containing an Fc region of an immunoglobulin.

The term “immunoglobulin,” “Ig” or “IgG” or “antibody” (used interchangeably herein) refers to a protein having a basic four-polypeptide chain structure consisting of two heavy and two light chains, said chains being stabilized, for example, by interchain disulfide bonds, which has the ability to specifically bind antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable,” based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions.” The “constant” domains of antibody light chains are referred to interchangeably as “light chain constant regions,” “light chain constant domains,” “CL” regions or “CL” domains. The “constant” domains of antibody heavy chains are referred to interchangeably as “heavy chain constant region,” “heavy chain constant domains,” “CH” regions or “CH” domains. The “variable” domains of antibody light chains are referred to interchangeably as “light chain variable regions,” “light chain variable domains,” “VL” regions or “VL” domains. The “variable” domains of antibody heavy chains are referred to interchangeably as “heavy chain variable regions.” “heavy chain variable domains,” “VH” regions or “VH” domains.

Immunoglobulins or antibodies may be monoclonal (referred to as a “MAb”) or polyclonal and may exist in monomeric or polymeric form, for example, IgM antibodies which exist in pentameric form and/or IgA antibodies which exist in monomeric, dimeric or multimeric form. Immunoglobulins or antibodies may also include multispecific antibodies (e.g., bispecific antibodies), and antibody fragments so long as they retain, or are modified to comprise, a ligand-specific binding domain. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. When produced recombinantly, fragments may be expressed alone or as part of a larger protein called a fusion protein. Exemplary fragments include Fab, Fab′, F(ab′)2, Fc and/or Fv fragments. Exemplary fusion proteins include Fc fusion proteins.

Generally, an immunoglobulin or antibody is directed against an “antigen” of interest. Preferably, the antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated. Where the antigen is a polypeptide, it may be a transmembrane molecule (e.g. receptor) or a ligand such as a growth factor.

The term “monoclonal antibody” or “MAb,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). “Monoclonal antibodies” may also be isolated from phage antibody libraries using the techniques described in Clackson et al., Nature 352:624-628 (1991) and Marks et al., J. Mol. Biol. 222:581-597 (1991), for example.

Monoclonal antibodies may further include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The term “hypervariable region” when used herein refers to the amino acid residues of an antibody which are responsible for antigen-binding. The hypervariable region comprises amino acid residues from a “complementarity determining region” or “CDR” (i.e. residues 24-34 (L1), 50-56 (L2) and 89-97 (L3) in the light chain variable domain and 31-35 (H1), 50-65 (1-12) and 95-102 (H3) in the heavy chain variable domain; Kabat et al., Sequences of Proteins of Immunological Interest. 5^{th} Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, an antibody which is separated or purified using a small molecule, as described herein, is a therapeutic antibody. Exemplary therapeutic antibodies include, for example, trastuzumab (HERCEPTIN™, Genentech, Inc., Carter et al (1992) Proc. Natl. Acad. Sci. USA, 89:4285-4289; U.S. Pat. No. 5,725,856); anti-CD20 antibodies such as chimeric anti-CD20 “C2B8” U.S. Pat. No. 5,736,137); rituximab (RITUXAN™), ocrelizumab, a chimeric or humanized variant of the 2H7 antibody (U.S. Pat. No. 5,721,108; WO 04/056312) or tositumomab (BEXXAR.™); anti-IL-8 (St John et al (1993) Chest, 103:932, and WO 95/23865); anti-VEGF antibodies including humanized and/or affinity matured anti-VEGF antibodies such as the humanized anti-VEGF antibody huA4.6.1 bevacizumab (AVASTIN™, Genentech, Inc., Kim et al (1992) Growth Factors 7:53-64, WO 96/30046, WO 98/45331); anti-PSCA antibodies (WO 01/40309); anti-CD40 antibodies, including S2C6 and humanized variants thereof (WO 00/75348); anti-CD11a (U.S. Pat. No. 5,622,700; WO 98/23761; Steppe et al (1991) Transplant Intl. 4:3-7; Hourmant et al (1994) Transplantation 58:377-380); anti-IgE (Presta et al (1993) J. Immunol. 151:2623-2632; WO 95/19181); anti-CD18 (U.S. Pat. No. 5,622,700; WO 97/26912); anti-IgE, including E25, E26 and E27 (U.S. Pat. No. 5,714,338; U.S. Pat. No. 5,091,313; WO 93/04173; U.S. Pat. No. 5,714,338); anti-Apo-2 receptor antibody (WO 98/51793); anti-TNF-alpha antibodies including cA2 (REMICADE™), CDP571 and MAK-195 (U.S. Pat. No. 5,672,347; Lorenz et al (1996) J. Immunol. 156(4):1646-1653; Dhainaut et al (1995) Crit. Care Med. 23(9):1461-1469); anti-Tissue Factor (TF) (EP 0 420 937 B1); anti-human alpha 4 beta 7 integrin (WO 98/06248); anti-EGFR, chimerized or humanized 225 antibody (WO 96/40210); anti-CD3 antibodies such as OKT3 (U.S. Pat. No. 4,515,893); anti-CD25 or anti-tac antibodies such as CHI-621 SIMULECT™ and ZENAPAX™ (U.S. Pat. No. 5,693,762); anti-CD4 antibodies such as the cM-7412 antibody (Choy et al (1996) Arthritis Rheum 39(1):52-56); anti-CD52 antibodies such as CAMPATH-1H-(Riechmann et al (1988) Nature 332:323-337); anti-Fc receptor antibodies such as the M22 antibody directed against Fc gamma RI as in Graziano et al (1995) J. Immunol. 155(10):4996-5002; anti-carcinoembryonic antigen (CEA) antibodies such as hMN-14 (Sharkey et al (1995) Cancer Res. 55(23Suppl): 5935s-5945s; antibodies directed against breast epithelial cells including huBrE-3, hu-Mc 3 and CHL6 (Ceriani et al (1995) Cancer Res. 55(23):5852s-5856s; and Richman et al (1995) Cancer Res. 55(23 Supp): 5916s-5920s); antibodies that bind to colon carcinoma cells such as C242 (Litton et al (1996) Eur J. Immunol. 26(1):1-9); anti-CD38 antibodies, e.g. AT 13/5 (Ellis et al (1995) J. Immunol. 155(2):925-937); anti-CD33 antibodies such as Hu M195 (Jurcic et al (1995) Cancer Res 55(23 Suppl):5908s-5910s and CMA-676 or CDP771; anti-CD22 antibodies such as LL2 or LymphoCide (Juweid et al (1995) Cancer Res 55(23 Suppl):5899s-5907s); anti-EpCAM antibodies such as 17-1A (PANOREX™); anti-GpIIb/IIIa antibodies such as abciximab or c7E3 Fab (REOPRO™); anti-RSV antibodies such as MEDI-493 (SYNAGIS™); anti-CMV antibodies such as PROTOVIR™); anti-HIV antibodies such as PRO542; anti-hepatitis antibodies such as the anti-Hep B antibody OSTAVIR™); anti-CA 125 antibody OvaRex; anti-idiotypic GD3 epitope antibody BEC2; anti-alpha v beta3 antibody VITAXIN™; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); and anti-human leukocyte antigen (HLA) antibodies such as Smart ID10 and the anti-HLA DR antibody Oncolym (Lym-1).

The terms “contaminant,” “impurity,” and “debris,” as used interchangeably herein, refer to any foreign or objectionable material, including a biological macromolecule such as a DNA, an RNA, one or more host cell proteins (HCPs or CHOPs), whole cells, cell debris and cell fragments, endotoxins, viruses, lipids and one or more additives which may be present in a sample containing a protein or polypeptide of interest (e.g., an antibody) being separated from one or more of the foreign or objectionable molecules using a non-polar and charged small molecule, as described herein.

In some embodiments according to the methods described herein, a small molecule comprising at least one non-polar group and at least one cationic group binds and precipitates an insoluble impurity (e.g. whole cells) present in a sample along with the protein of interest, thereby to separate the protein of interest from such an impurity. In other embodiments according to the methods described herein, a small molecule comprising at least one anionic group and at least one non-polar group binds and precipitates a protein or polypeptide of interest, thereby to separate it from one or more impurities (e.g., soluble impurities).

The term “insoluble impurity,” as used herein, refers to any undesirable or objectionable entity present in a sample containing a target biomolecule, wherein the entity is a suspended particle or a solid. Exemplary insoluble impurities include whole cells, cell fragments and cell debris.

The term “soluble impurity,” as used herein, refers to any undesirable or objectionable entity present in a sample containing a target biomolecule, wherein the entity is not an insoluble impurity. Exemplary soluble impurities include host cell proteins, DNA, RNA, viruses, endotoxins, cell culture media components, lipids etc.

The term “composition,” “solution” or “sample,” as used herein, refers to a mixture of a target biomolecule or a product of interest to be purified along with one or more undesirable entities or impurities. In some embodiments, the sample comprises a biological material containing stream, e.g., feedstock or cell culture media into which a target biomolecule or a desired product is secreted. In some embodiments, the sample comprises a target biomolecule (e.g., a therapeutic protein or an antibody) along with one or more soluble and/or insoluble impurities (e.g., host cell proteins, DNA, RNA, lipids, cell culture additives, endotoxins, whole cells and cellular debris). In some embodiments, the sample comprises a target biomolecule which is secreted into the cell culture media. The target biomolecule may be separated from one or more undesirable entities or impurities either by precipitating the one or more impurities or by precipitating the target molecule.

In some embodiments, a small molecule according to the present invention binds to a target biomolecule or product (e.g., a target protein or polypeptide), where the small molecule comprises at least one anionic group and at least one non-polar group. This process may be referred to as “capture.” Exemplary small molecules comprising at least one anionic group and at least one non-polar group include, but are not limited to, pterin derivatives (for example folic acid, pteroic acid), etacrynic acid, fenofibric acid, mefenamic acid, mycophenolic acid, tranexamic acid, zoledronic acid, acetylsalicylic acid, arsanilic acid, ceftiofur acid, meclofenamic acid, ibuprofine, naproxen, fusidic acid, nalidixic acid, chenodeoxycholic acid, ursodeoxycholic acid, tiaprofenic acid, niflumic acid, trans-2-hydroxycinnamic acid, 3-phenylpropionic acid, probenecid, clorazepate, icosapent, 4-acetamidobenzoic acid, ketoprofen, tretinoin, adenylosuccinic acid, naphthalene-2,6-disulfonic acid, tamibarotene, etodolacetodolic acid, and benzylpenicillinic acid.

Additional exemplary small molecules having at least one anionic group and at least one non-polar group include, but are not limited to, dye molecules, e.g., Amaranth and Nitro red.

In other embodiments, methods for separating a biomolecule of interest from one or more impurities employ a small molecule which binds to the one or more impurities (e.g., insoluble impurities). Such a process may be referred to as “clarification.” In some embodiments, such small molecules include at least one cationic group and at least one non-polar group. Exemplary small molecules that may be used for clarification include, but are not limited to, monoalkyltrimethyl ammonium salt (non-limiting examples include cetyltrimethylammonium bromide or chloride, tetradecyltrimethylammonium bromide or chloride, alkyltrimethyl ammonium chloride, alkylaryltrimethyl ammonium chloride, dodecyltrimethylammonium bromide or chloride, dodecyldimethyl-2-phenoxyethylammonium bromide, hexadecylamine chloride or bromide, dodecyl amine or chloride, and cetyldimethylethyl ammonium bromide or chloride), a monoalkyldimethylbenzyl ammonium salt (non-limiting examples include alkyldimethylbenzyl ammonium chloride and benzethonium chloride), a dialkyldimethyl ammonium salt (non-limiting examples include domiphen bromide, didecyldimethyl ammonium halides (bromide and chloride salts) and octyldodecyldimethyl ammonium chloride or bromide), a heteroaromatic ammonium salt (non-limiting examples include cetylpyridium halides (chloride or bromide salts) and hexadecylpyridinium bromide or chloride, cis-isomer 1-[3-chloroallyl]-3,5,7-triaza-1-azoniaadamantane, alkyl-isoquinolinium bromide, and alkyldimethylnaphthylmethyl ammonium chloride), a polysubstituted quaternary ammonium salt, (non-limiting examples include alkyldimethylbenzyl ammonium saccharinate and alkyldimethylethylbenzyl ammonium cyclohexylsulfamate), and a bis-quaternary ammonium salt (non-limiting examples include 1,10-bis(2-methyl-4-aminoquinolinium chloride)-decane, 1,6-Bis{1-methyl-3-(2,2,6-trimethyl cyclohexyl)-propyldimethyl ammonium chloride]hexane or triclobisonium chloride, and the bis-quat referred to as CDQ by Buckman Brochures).

The term “precipitate,” precipitating” or “precipitation,” as used herein, refers to the alteration of a bound (e.g. in a complex with a biomolecule of interest) or unbound small molecule from an aqueous and/or soluble state to a non-aqueous and/or insoluble state. The precipitate is also referred to as a solid or a solid phase.

The terms “chinese hamster ovary cell protein” and “CHOP,” as used interchangeably herein, refer to a mixture of host cell proteins (“HCP”) derived from a Chinese hamster ovary (“CHO”) cell culture. The HCP or CHOP is generally present as a soluble impurity in a cell culture medium or lysate (e.g., a harvested cell culture fluid containing a protein or polypeptide of interest (e.g., an antibody or immunoadhesin expressed in a CHO cell). Generally, the amount of CHOP present in a mixture comprising a protein of interest provides a measure of the degree of purity for the protein of interest. Typically, the amount of CHOP in a protein mixture is expressed in parts per million relative to the amount of the protein of interest in the mixture.

It is understood that where the host cell is another mammalian cell type, an E. coli, a yeast cell, an insect cell, or a plant cell, HCP refers to the proteins, other than target protein, found in a lysate of the host cell.

The term “cell culture additive,” as used herein, refers to a molecule (e.g., a non-protein additive), which is added to a cell culture process in order to facilitate or improve the cell culture or fermentation process. In some embodiments according to the present invention, a small molecule, as described herein, binds and precipitates one or more cell culture additives. Exemplary cell culture additives include anti-foam agents, antibiotics, dyes and nutrients.

The term “parts per million” or “ppm,” as used interchangeably herein, refers to a measure of purity of a desired target molecule (e.g., a target protein or antibody) purified using a small molecule, as described herein. Accordingly, this measure can be used either to gauge the amount of a target molecule present after the purification process or to gauge the amount of an undesired entity.

The terms “isolating,” “purifying” and “separating,” are used interchangeably herein, in the context of purifying a target biomolecule (e.g., a polypeptide or a protein of interest) from a composition or sample comprising the target biomolecule and one or more impurities, using a small molecule, as described herein. In some embodiments, the degree of purity of the target biomolecule in a sample is increased by removing (completely or partially) one or more insoluble impurities (e.g., whole cells and cell debris) from the sample by using a small molecule comprising at least one non-polar group and at least one cationic group, as described herein. In another embodiment, the degree of purity of the target biomolecule in a sample is increased by precipitating the target biomolecule away from one or more soluble impurities in the sample, e.g., by using a small molecule comprising an anionic group and a non-polar group.

In some embodiments, a purification process additionally employs one or more “chromatography steps.” Typically, these steps may be carried out, if necessary, after the separation of a target biomolecule from one or more undesired entities using a small molecule, as described herein.

In some embodiments, a “purification step” to isolate, separate or purify a polypeptide or protein of interest using a small molecule, as described herein, may be part of an overall purification process resulting in a “homogeneous” or “pure” composition or sample, which term is used herein to refer to a composition or sample comprising less than 100 ppm HCP in a composition comprising the protein of interest, alternatively less than 90 ppm, less than 80 ppm, less than 70 ppm, less than 60 ppm, less than 50 ppm, less than 40 ppm, less than 30 ppm, less than 20 ppm, less than 10 ppm, less than 5 ppm, or less than 3 ppm of HCP.

The term “clarification,” or “clarification step,” as used herein, generally refers to one or more initial steps in the purification of biomolecules. The clarification step generally comprises removal of whole cells and/or cellular debris using one or more steps including any of the following alone or various combinations thereof, e.g., centrifugation and depth filtration, precipitation, flocculation and settling. Clarification step generally involves the removal of one or more undesirable entities and is typically performed prior to a step involving capture of the desired target molecule. Another key aspect of clarification is the removal of insoluble components in a sample which may later on result in the fouling of a sterile filter in a purification process, thereby making the overall purification process more economical. In some embodiments, the present invention provides an improvement (e.g., requirement of less filter area used downstream) over the conventional clarification steps commonly used, e.g., depth filtration and centrifugation.

The term “chromatography,” as used herein, refers to any kind of technique which separates an analyte of interest (e.g., a target biomolecule) from other molecules present in a mixture. Usually, the analyte of interest is separated from other molecules as a result of differences in rates at which the individual molecules of the mixture migrate through a stationary medium under the influence of a moving phase, or in bind and elute processes.

The term “chromatography resin” or “chromatography media” are used interchangeably herein and refer to any kind of phase (e.g., a solid phase) which separates an analyte of interest (e.g., a target biomolecule) from other molecules present in a mixture. Usually, the analyte of interest is separated from other molecules as a result of differences in rates at which the individual molecules of the mixture migrate through a stationary solid phase under the influence of a moving phase, or in bind and elute processes. Examples of various types of chromatography media include, for example, cation exchange resins, affinity resins, anion exchange resins, anion exchange membranes, hydrophobic interaction resins and ion exchange monoliths.

The term “capture step” or “capture,” as used herein, generally refers to a method used for binding a target biomolecule with a small molecule, in a quantity and under conditions suitable to precipitate the target biomolecule. Typically, the target biomolecule is subsequently recovered by reconstitution of the precipitate into a suitable buffer. In some embodiments according to the methods described herein, a target biomolecule is captured using a small molecule comprising at least one anionic group and at least one non-polar group, which may be aromatic or aliphatic.

The term “process step” or “unit operation,” as used interchangeably herein, refers to the use of one or more methods or devices to achieve a certain result in a purification process. One or more process steps or unit operations in a purification process may employ one or more small molecules encompassed by the present invention. Examples of process steps or unit operations which may be employed in the processes described herein include, but are not limited to, °clarification, bind and elute chromatography, virus inactivation, flow-through purification and formulation. In some embodiments, one or more devices which are used to perform a process step or unit operation are single-use devices and can be removed and/or replaced without having to replace any other devices in the process or even having to stop a process run. In some embodiments, one or more small molecules are used to remove one or more impurities during a clarification step of a purification process.

The term “surge tank” as used herein refers to any container or vessel or bag, which is used between process steps or within a process step (e.g. when a single process step comprises more than one step); where the output from one step flows into the surge tank and onto the next step. Accordingly, a surge tank is different from a pool tank, in that it is not intended to hold or collect the entire volume of output from a step; but instead enables continuous flow of output from one step to the next, as liquid may be pumped into and out of the surge tank. In some embodiments, the volume of a surge tank used between two process steps or within a process step in a process or system described herein, is no more than 25% of the entire volume of the output from the process step. In another embodiment, the volume of a surge tank is no more than 10% of the entire volume of the output from a process step. In some other embodiments, the volume of a surge tank is less than 35%, or less than 30%, or less than 25%, or less than 20%, or less than 15%, or less than 10% of the entire volume of a cell culture in a bioreactor, which constitutes the starting material from which a target molecule is to be purified.

The term “continuous process,” as used herein, refers to a process for purifying a target molecule, which includes two or more process steps (or unit operations), such that the output from one process step flows directly into the next process step in the process, without interruption, and where two or more process steps can be performed concurrently for at least a portion of their duration. In other words, in case of a continuous process, as described herein, it is not necessary to complete a process step before the next process step is started, but a portion of the sample is always moving through the process steps. The term “continuous process” also applies to steps within a process step, in which case, during the performance of a process step including multiple steps, the sample flows continuously through the multiple steps that are necessary to perform the process step. In some embodiments, the small molecules described herein are used in a purification process which is performed in a continuous mode, such the output from one step flows into the next step without interruption, where the two steps are performed concurrently for at least portion of their duration. In a particular embodiment, a small molecule is used for clarification, as described herein, following which process step, the output containing the target molecule directly flows onto the next step (e.g., an affinity chromatography step). In some embodiments, centrifugation or filtration may be used following clarification and before affinity chromatography.

The term “static mixer” refers to a device for mixing two fluid materials, typically liquids. The device generally consists of mixer elements contained in a cylindrical (tube) housing. The overall system design incorporates a method for delivering two streams of fluids into the static mixer. As the streams move through the mixer, the non-moving elements continuously blend the materials. Complete mixing depends on many variables including the properties of the fluids, inner diameter of the tube, number of mixer elements and their design etc. In some embodiments described herein, one or more static mixers are used throughout the purification process. In a particular embodiment, a static mixer may be used for mixing one or more small molecules with a sample feed stream. Accordingly, in some embodiments, one or more small molecules are added to a sample feed stream in a continuous manner, e.g., using a static mixer.

### II. EXEMPLARY SMALL MOLECULES COMPRISING AT LEAST ONE NON-POLAR GROUP AND AT LEAST ONE CATIONIC GROUP

In some embodiments, the present invention relates to a method of separating a target biomolecule from one or more insoluble impurities in a sample and employs small molecules that include at least one non-polar group and at least one cationic group, which bind to and precipitate one or more impurities (e.g., insoluble impurities), thereby separating the target biomolecule from such impurities. The non-polar group may be aromatic or aliphatic.

Non-limiting examples of small molecules having at least one non-polar group and at least one cationic group include, but are not limited to, a monoalkyltrimethyl ammonium salt (e.g., cetyltrimethylammonium bromide, cetyltrimethylammonium chloride, tetradecyltrimethylammonium bromide, tetradecyltrimethylammonium chloride, alkyltrimethyl ammonium chloride, alkylaryltrimethyl ammonium chloride, dodecyltrimethylammonium bromide, dodecyltrimethylammonium chloride, dodecyldimethyl-2-phenoxyethylammonium bromide, hexadecylamine chloride, hexadecylamine bromide, dodecyl amine, dodecyl chloride, cetyldimethylethyl ammonium bromide and cetyldimethylethyl ammonium chloride), a monoalkyldimethylbenzyl ammonium salt (e.g., alkyldimethylbenzyl ammonium chloride and benzethonium chloride), a dialkyldimethyl ammonium salt (e.g., domiphen bromide, didecyldimethyl ammonium chloride, didecyldimethyl ammonium bromide, octyldodecyldimethyl ammonium chloride and octyldodecyldimethyl ammonium bromide), a heteroaromatic ammonium salt (e.g., cetylpyridium chloride, cetylpyridium bromide, hexadecylpyridinium bromide, hexadecylpyridinium chloride, cis-isomer 1-[3-chloroallyl]-3,5,7-triaza-1-azoniaadamantane, alkyl-isoquinolinium bromide, and alkyldimethylnaphthylmethyl ammonium chloride), a polysubstituted quaternary ammonium salt (e.g., alkyldimethylbenzyl ammonium saccharinate, and alkyldimethylethylbenzyl ammonium cyclohexylsulfamate) and a bis-quaternary ammonium salt (e.g., 1,10-bis(2-methyl-4-aminoquinolinium chloride)-decane, 1,6-Bis{1-methyl-3-(2,2,6-trimethyl cyclohexyl)-propyldimethyl ammonium chloride]hexane or triclobisonium chloride, and the bis-quat, referred to as CDQ by Buckman Brochures).

In a particular embodiment, a small molecule comprising a non-polar group and a cationic group is benzethonium chloride (BZC).

In some embodiments, such small molecules are used during the clarification process step of a purification process.

### III. EXEMPLARY SMALL MOLECULES COMPRISING AT LEAST ONE NON-POLAR GROUP AND AT LEAST ONE ANIONIC GROUP

In some embodiments, the present invention relates to a method of purifying a target biomolecule from a sample comprising the target molecule along with one or more impurities (e.g., soluble impurities), where the method employs the use of a small molecule which includes at least one anionic group and at least one non-polar group. The non-polar group may be aromatic or aliphatic. In some embodiments, the small molecule comprises a non-polar group which is aromatic. In other embodiments, the small molecule comprises a non-polar group which is aliphatic.

Exemplary small molecules comprising at least one anionic group and at least one non-polar group include, but are not limited to, pterin derivatives (for example folic acid, pteroic acid), etacrynic acid, fenofibric acid, mefenamic acid, mycophenolic acid, tranexamic acid, zoledronic acid, acetylsalicylic acid, arsanilic acid, ceftiofur acid, meclofenamic acid, ibuprofine, naproxen, fusidic acid, nalidixic acid, chenodeoxycholic acid, ursodeoxycholic acid, tiaprofenic acid, niflumic acid, trans-2-hydroxycinnamic acid, 3-phenylpropionic acid, probenecid, clorazepate, icosapent, 4-acetamidobenzoic acid, ketoprofen, tretinoin, adenylosuccinic acid, naphthalene-2,6-disulfonic acid, tamibarotene, etodolacetodolic acid and benzylpenicillinic acid.

In a particular embodiment, a small molecule including at least one anionic group and at least one non-polar group is folic acid or a derivative thereof.

Also encompassed by the present invention are certain dye molecules which may be used for binding and precipitating a target biomolecule. Examples include, but are not limited to, Amaranth and Nitro red.

### IV. PROTEIN PURIFICATION METHODS EMPLOYING SMALL MOLECULES

In the various methods encompassed by the present invention, a small molecule is added at one or more stages of a protein purification process, thereby to precipitate one or more impurities or to precipitate the target biomolecule.

One such exemplary process employs contacting a cell culture feed containing a target biomolecule and one or more impurities with a suitable amount of a small molecule including at least one non-polar group and at least one cationic group (e.g., 0.4% wt of BZC), thereby to precipitate one or more impurities (e.g., insoluble impurities). The solid phase of the sample (i.e., containing the precipitate) can be removed by depth filtration or centrifugation. The remaining sample containing the target biomolecule can then be subjected to subsequent purification steps (e.g., one or more chromatography steps).

In another exemplary process according to the present invention, a small molecule is added at one or more steps of a protein purification process, where the small molecule binds and precipitates the target biomolecule itself. Such a small molecule includes at least one non-polar group and at least one anionic group.

Generally, a cell culture feed is subjected to a clarification step prior to contacting it with the small molecule including at least one anionic group and at least one non-polar group. The clarification step is intended to remove the insoluble impurities. For example, in an exemplary method described herein, a clarified cell culture feed containing a target molecule and one or more soluble impurities is contacted with a suitable amount of a small molecule including an anionic group and a non-polar group (e.g., 1:1 mass ratio of folic acid). The sample is then subjected to a change in pH conditions thereby to facilitate the precipitation of the target biomolecule (e.g., changing pH to pH 5.0 using acetic acid). The precipitate, which contains the target biomolecule is subsequently washed with a suitable buffer (e.g., 0.1 M arginine at pH 5.0) and the target biomolecule is subsequently resolubilized using a suitable buffer (0.1M thiamine at pH 7.0). Any residual amounts of the small molecule (e.g., folic acid) in the solution with the resolubilized target biomolecule can be subsequently removed using suitable means (e.g., activated carbon). The target biomolecule containing solution is typically subjected to additional polishing steps in order to recover a significantly pure sample of the target biomolecule.

In some other embodiments according to the claimed invention, different types of small molecules (e.g., those which bind the one or more impurities and those which bind the target biomolecule) are both used in different steps of the same protein purification process. For example, a small molecule including at least one cationic group and at least one non-polar group (e.g., BZC) can be used in the clarification step to remove the one or more insoluble impurities, and the target biomolecule in the same sample can be then precipitated using a small molecule including at least one anionic group and at least one non-polar group (e.g., folic acid).

As discussed above, residual amounts of small molecules remaining in a sample containing a target biomolecule can be subsequently removed using suitable materials such as, for example, activated carbon. The sample is generally subjected to additional chromatography or non-chromatography steps to achieve desirable levels of product purity.

In some embodiments, one or more small molecules described herein are used in a purification process which is performed in a continuous format. In such a purification process, several steps may be employed, including, but not limited to, e.g., culturing cells expressing protein in a bioreactor; subjecting the cell culture to clarification, which may employ the use of one or more small molecules described herein, and optionally using a depth filter; transferring the clarified cell culture to a bind and elute chromatography capture step (e.g., Protein A affinity chromatography); subjecting the Protein A eluate to virus inactivation (e.g., using one or more static mixers and/or surge tanks); subjecting the output from virus inactivation to a flow-through purification process, which employs two or more matrices selected from activated carbon, anion exchange chromatography media, cation exchange chromatography media and virus filtration media; and formulating the protein using diafiltration/concentration and sterile filtration. Additional details of such processes can be found, e.g. in co-pending application having reference no. P12/107, filed concurrently herewith, the entire contents of which are incorporated by reference herein.

This invention is further illustrated by the following examples which should not be construed as limiting. The contents of all references, patents and published patent applications cited throughout this application, as well as the Figures, are incorporated herein by reference.

### EXAMPLES

### Example 1

### Preparation of Expressing Cell Culture Fluid (CCF)

In a representative experiment, cells derived from a Chinese Hamster Ovary (CHO) cell line expressing a monoclonal IgG₁ were grown in a 10 L bioreactor (NEW BRUNSWICK SCIENTIFIC) to a density of 13×10⁶ cells/mL and harvested at <50% viability. The antibody titer was determined in the range of 0.85-1.8 mg/mL via protein A HPLC. The level of host cell proteins (HCP) was found to be 350000-425000 ng/mL using an ELISA (CYGNUS #F550). The pH of the unclarified cell culture was pH 7.2.

### Example 2

### Preparation of Expressing Clarified Cell Culture Fluid (CCF)

Feed from Example 1 was clarified by centrifugation at 4000 rpm for 2 min, followed by filtration through Sum and 0.2 μm Durapore® filters.

### Example 3

### Preparation of Non-Expressing Cell Culture Fluid (CCF)

In another experiment, cells derived from a non-IgG-expressing Chinese Hamster Ovary (CHO) cell line were grown in a 10 L bioreactor (NEW BRUNSWICK SCIENTIFIC) to a density of 13×10⁶ cells/mL and harvested at <50% viability. The level of host cell proteins (HCP) was found to be 66000-177000 ng/mL using an ELISA (CYGNUS #F550). The pH of the unclarified cell culture was pH 7.2.

### Example 4

### Preparation of Non-Expressing Clarified Cell Culture Fluid (CCF)

Feed from Example 3 was clarified by centrifugation at 4000 rpm for 2 min, followed by filtration through 5 μm and 0.2 μm Durapore® filters.

### Example 5

### Preparation of Clarified Cell Culture Fluid (CCF) with IgG Spike

Feed from Example 4 was spiked with pure IgG₁ purified using Prosep ultra plus (EMD Millipore) protein A resin. The final concentration of IgG was ˜1 g/L as determined using Protein A HPLC (Agilent Technologies).

### Example 6

### Preparation of Benzethonium Chloride (BZC) Solution

A 100 g/L solution of Benzethonium Chloride (BZC) (≧97%, Sigma-Aldrich), was prepared by dissolving 100 g in 1 L deionized water with continued mixing for 30 min at room temperature.

### Example 7

### Preparation of Hexadecyltrimethylammonium Bromide Solution

A 40 g/L solution of Hexadecyltrimethylammonium bromide, (>98%, Sigma-Aldrich), HTAB, was prepared by dissolving 40 g in 1 L phosphate buffered saline (PBS).

### Example 8

### Preparation of Sodium Tetrafluoroborate Solution

A 5 g/L solution of Sodium Tetrafluoroborate (98%, Sigma-Aldrich), was prepared by dissolving 5 g in 1 L deionized water with continued mixing for 30 min at room temperature.

### Example 9

### Preparation of Folic Acid Solution

A 80 g/L solution of Folic acid (>97%, Sigma-Aldrich), FA, was prepared by dissolving 80 g in 1 L of 0.4M Sodium hydroxide with continued mixing for 60 min at room temperature. The final solution pH was around 8. The solution was then filtered through 0.2 μm Durapore® filter to remove any remaining un-dissolved solid. The color of the solution was dark brown.

### Example 10

### Preparation of Amaranth Solution

A 50 g/L solution of Amaranth (>98%, Sigma-Aldrich), was prepared by dissolving 50 g in 1 L of 20 mM sodium acetate, pH 4.5 with continued mixing for 30 min at room temperature. The final solution pH was around 4.5. The solution was then filtered through 0.2 μm Durapore® filter to remove any remaining un-dissolved solid. The color of the solution was dark red.

### Example 11

### Preparation of Nitro-Red Solution

A 50 g/L solution of Nitro red (4-Amino-5-hydroxy-3-(4-nitrophenylazo)-2,7-naphthalenedisulfonic acid disodium salt (>98%, Sigma-Aldrich), was prepared by dissolving 50 g in 1 L of 20 mM sodium acetate, pH 4.0 with continued mixing for 30 min at room temperature. The final solution pH was around 4.0. The solution was then filtered through 0.2 μm Durapore® filter to remove any remaining un-dissolved solid. The color of the solution was dark red.

### Example 12

### Generation of a Calibration Curve for Detection of BZC in Solution

In a representative experiment described herein, a turbidimetric assay was used to generate a calibration curve which was used for the detection of amounts of BZC in solution.

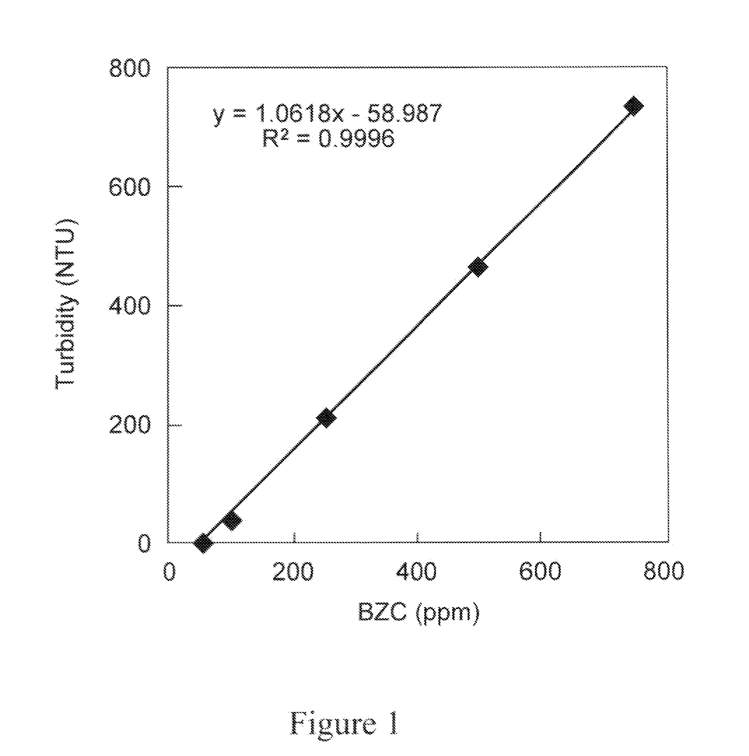

A series of BZC solutions at 750, 500, 250, 100, and 50 mg/L were prepared in deionized water by serial dilutions starting from the stock solution described in Example 6. To 5 ml of each of the diluted BZC solutions, 5 ml of solution from Example 8 was added and continuously mixed at room temperature for 10 min. The solutions turned turbid upon mixing due to complexation between BZC and sodium tetrafloroborate. The turbidity of the solutions was measured using a 2100p turbidimeter (HACH Company, Colorado USA) and used to generate a calibration curve, depicted in FIG. 1.

The limit of detection of this assay is 100 mg/L BZC in solution. The calibration curve was used to quantify residual amounts of BZC in BZC clarified feeds.

### Example 13

### Removal of BZC from Solution

In a representative experiment described herein, certain materials (i.e., activated carbon) were shown to be useful for the removal of BZC from solution. Such materials can be used for removing BZC in a sample containing a target biomolecule following precipitation of insoluble impurities using BZC.

A 5 ml BZC solution (5 mg/ml), prepared by mixing 0.25 ml of solution from Example 6 with 4.75 ml of deionized water, was mixed with 0.05, 0.1, 0.15 and 0.2 g of activated carbon (NUCHER SA-20, Meadwestvaco, Covington, Va.) for 10 min at room temperature. The activated carbon was then collected by centrifugation (4000 rpm for 2 min) and the supernatant filtered through 5 and 0.2μ Millex® filters available from Millipore Corporation of Billerica, Mass. Using the turbidimetric assay outlined in Example 12, the amount of BZC left in solution after treatment with activated carbon was determined.

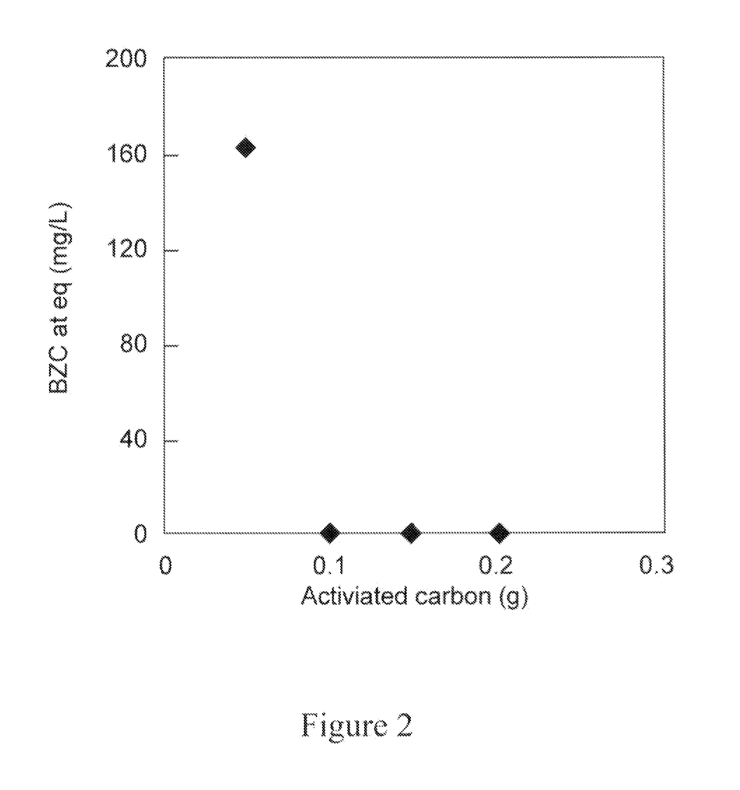

As depicted in FIG. 2, 0.1 g of carbon is enough to reduce 25 mg BZC in solution to an undetected level (less than 100 mg/L). This information was later utilized to estimate the amount of activated carbon suitable to remove residual amounts of BZC from BZC clarified cell culture media.

### Example 14

### Clarification of Cell Culture Media and Subsequent Clearance of HCP Using BZC

In a representative experiment described herein. BZC was used for removal of insoluble impurities from a sample containing a target biomolecule of interest, which was an IgG 1 monoclonal antibody (MAb) molecule. Subsequent to the use of BZC for clarification, as described herein, activated carbon may be used for removing residual amounts of BZC from the sample.

1.6 ml of BZC from Example 6 was added to 40 ml of the un-clarified feed from Example 1 (1.8 g/L IgG 1) and mixed at room temperature for 10 minutes, to allow for binding and precipitation of impurities. The supernatant was then separated from the precipitate by centrifugation (4000 rpm for 1 min).

To determine the residual amount of BZC that remained in solution, a 5 ml sample of the supernatant were mixed with 5 ml of Sodium Tetrafluoroborate solution (from Example 8) for 10 minutes at room temperature. The resulting turbidity, measured on 2100p turbidimeter (HACH Company, Colorado, USA), corresponded to 512 mg/L residual BZC (using the calibration curve from Example 12).

Residual BZC in solution was removed from the remaining 36 ml of supernatant by adding 1.2 g of activated carbon (NUCHER SA-20, Meadwestvaco, Covington, Va.) with continuous mixing at room temperature for 5 min. The amount of activated carbon was added in excess of what is needed per Example 13 (i.e, 0.072 g of activated carbon), in order to decrease the concentration of residual BZC in solution below the detection limit. Since media components can also bind to activated carbon, the latter had to be added in excess such that activated carbon has some capacity left to bind residual BZC in solution. The activated carbon was then collected by centrifugation (4000 rpm for 2 min) and the supernatant filtered through 0.2 g Durapore® filter.

Under these conditions. ˜90% of the IgG present in the original fluid was recovered, 94% of the HCP was removed and residual BZC in solution was below the detection limit.

### Example 15

### Optimization of Impurity Clearance Using Clarification with BZC

In a representative experiment described herein, the optimal concentration of BZC for maximum recovery of a target biomolecule (e.g., a monoclonal antibody (MAb) molecule) as well as maximum impurity clearance was determined.

0.8, 1.6, 2.4 ml of BZC from Example 6 was added to 40 ml of the un-clarified feed from Example 1 (1.8 g/L IgG₁) and mixed at room temperature for 10 minutes, to allow for binding and precipitation of impurities. The precipitate was then collected by centrifugation (4000 rpm for 1 min) and the supernatant was further purified to remove excess residual BZC by adding 1.2 g of activated carbon (NUCHER SA-20. Meadwestvaco, Covington, Va.) with continuous mixing at room temperature for 5 min. The activated carbon was then collected by centrifugation (4000 rpm for 2 min) and the supernatant filtered through 5 and 0.2μ Millex® filters available from Millipore Corporation of Billerica, Mass. The optimal BZC concentration was determined to be ˜4 g/L (1.6 ml of BZC from Example 6) which resulted in ˜90% HCP clearance and ˜94% MAb recovery.

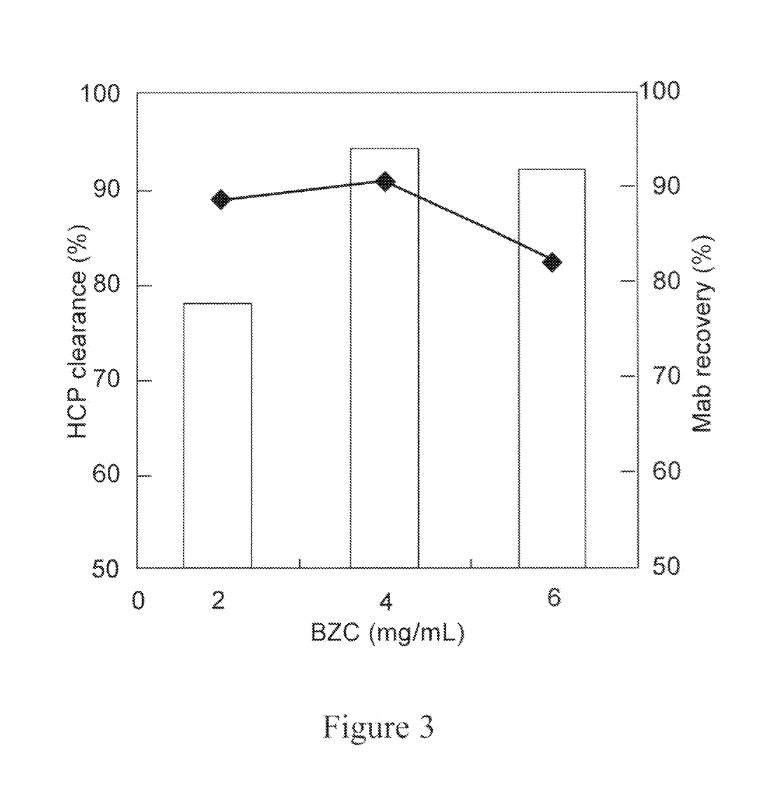

As shown in FIG. 3, ˜4 g/L BZC could be used for removal of most of the impurities without effecting MAb recovery.

### Example 16

### Identifying the Amount of Folic Acid Required to Precipitate MAb

In a representative experiment described herein, the amount of folic acid required for efficient MAb precipitation (90% or more) was determined.

4.75 ml of feed from Example 2 (1.1 g/L IgG₁) was mixed with different volumes of Folic acid from Example 9 and Dionized water as depicted in Table 1. The pH of the solution was adjusted to 4.5, 5.0, 5.5 and 6.0 using 3M acetic acid (Fisher Scientific) and continuously mixed at room temperature for 10 min. When a suitable folic acid to MAb ratio was reached, a precipitate, in the form of dispersed solid suspension, formed instantly as a result of folic acid complexing with MAb. The precipitate was then collected by centrifugation (4000 rpm for 1 min) and the supernatant filtered through 0.2 Durapore® filter.

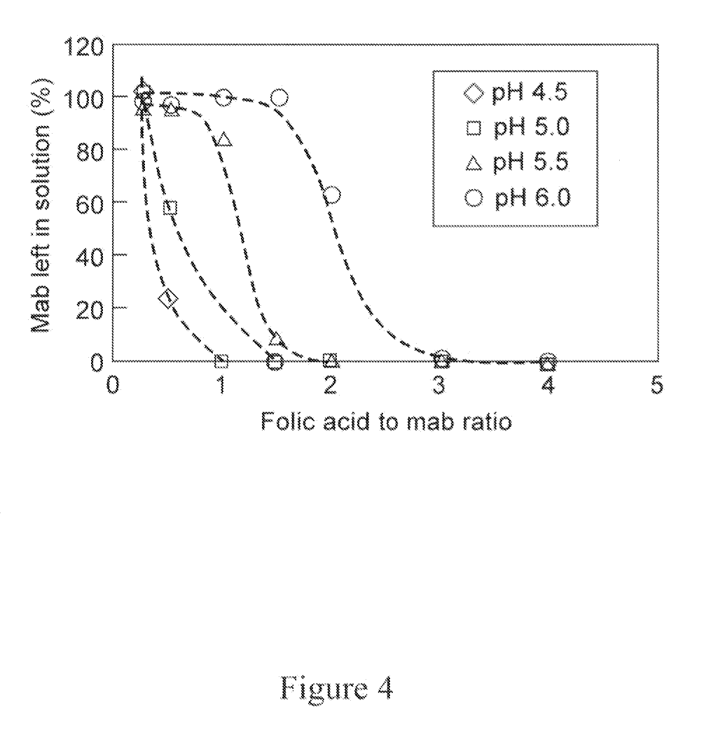

As depicted in FIG. 4, the amount of folic acid necessary to bind and precipitate IgG₁ with >90% efficiency increases as the solution pH increases.

TABLE 1

| Folic acid (ml) | Deionized water |
|---|---|
| 0.0125 | 0.238 |
| 0.0313 | 0.22 |
| 0.0625 | 0.188 |
| 0.094 | 0.156 |
| 0.125 | 0.125 |
| 0.188 | 0.063 |
| 0.25 | 0 |

### Example 17

### Capture of a Desired MAb Molecule from Clarified Cell Culture Media Using Folic Acid

In a representative experiment described herein, folic acid was used for capturing a MAb molecule from clarified CHO cell culture.

0.152 ml of folic acid from Example 9, and 0.098 ml of Deionized water were added to 4.75 ml of feed from Example 2 (1.8 g/L, IgG₁). The pH of the solution was adjusted 5.5 using 3M acetic acid and continuously mixed at room temperature for 10 min. After acid addition, a precipitate, in the form of dispersed solid suspension, formed instantly as a result of folic acid complexing with MAb. The precipitate was then collected by centrifugation (4000 rpm for 1 min) and washed with Tris buffer from Fisher Scientific (25 mM, pH 6.0) in order to remove loosely-bound impurities. Re-solubilization of the precipitate and elution of IgG took place at pH 7.5 using 25 mM Tris buffer containing 0.5M NaCl while mixing continuously for 10 min at room temperature. Removal of the free folic acid is effected by adding 50 mM CaCl₂ (Fisher Scientific), which precipitates folic acid, followed by filtration through 5 and 0.2 μm Millex® filters available from Millipore Corporation of Billerica, Mass. The purified MAb molecule is then recovered in the supernatant fluid.

Under these conditions, >95% of the MAb present in the original fluid bound to folic acid and 88% of IgG was recovered upon elution.

### Example 18

### Level of HCP in a Solution Containing Folic Acid Captured MAb

Following the capture of the MAb molecule using folic acid, as described in Example 17, the level of HCP was measured in the sample containing the MAb. An ELISA assay kit (CYGNUS #F550) was used to track the level of host cell protein (HCP) at different steps of the product (IgG) capture process. The concentration of HCP was reduced from 424306 ng/ml in the starting cell culture fluid to 146178 ng/ml in the elution sample, thereby demonstrating a reduction in HCP levels by 65%.

### Example 19

### Generation of a Calibration Curve to Determine the Concentration of Folic Acid in Solution

In a representative experiment described herein, a calibration curve was generated in order to subsequently quantify the amounts of residual folic acid remaining in solution.

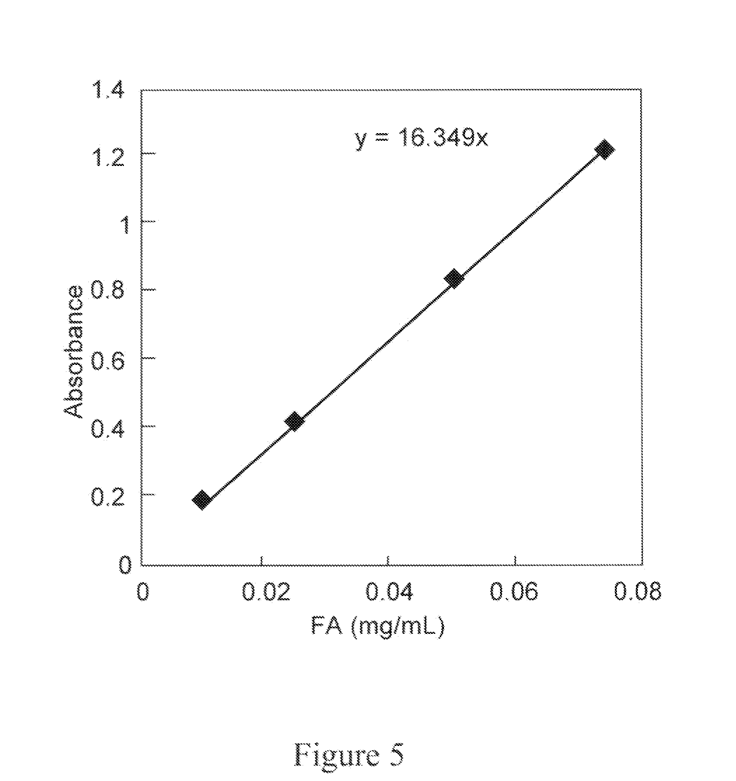

Standard solutions of folic acid at 0.01, 0.025, 0.05 and 0.075 mg/ml were prepared in deionized water by serial dilutions of the folic acid solution from Example 9. The absorbance of the standard solutions was measured at 350 nm using a spectrophotometer, and a standard curve was plotted, as depicted in FIG. 5.

### Example 20

### Removal of Folic Acid with Activated Carbon

In a representative experiment, it was demonstrated that certain materials such as, for example activated carbon, can be used for removing folic acid from solution

0.75, 2.4, 4.85, 8.2 and 12.9 mg/ml of folic acid solutions were prepared in 0.1 M Thiamine hydrochloride at pH 7 (Sigma) by serial dilution of folic acid solution from Example 9. The solutions were mixed with 0.5 g of activated carbon (NUCHER SA-20, Meadwestvaco, Covington, Va.) with continuous mixing at room temperature for 10 min. The activated carbon was then collected by centrifugation (4000 rpm for 2 min) and the supernatant filtered through 5 and 0.2μ Millex® filters available from Millipore Corporation of Billerica, Mass. The concentration of folic acid left in solution was determined by measuring absorbance at 350 nm and using the calibration curve described in Example 19.

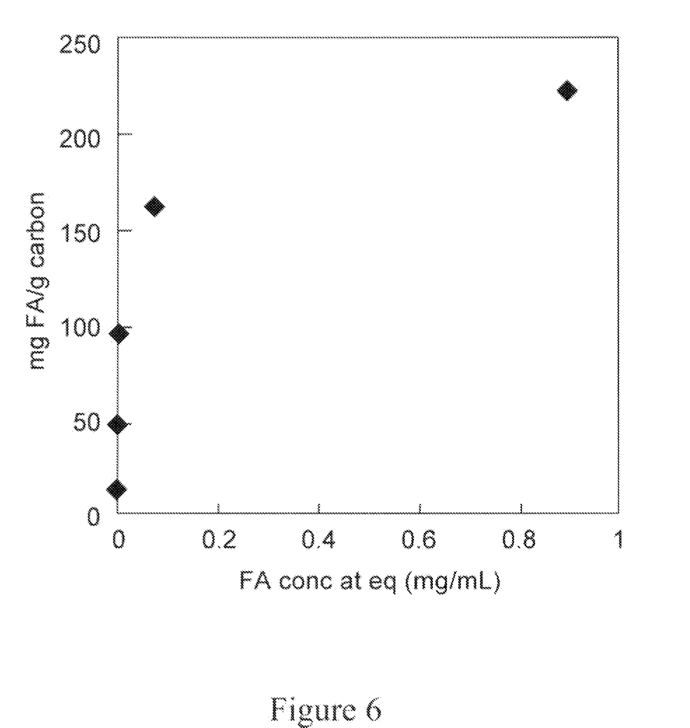

As depicted in FIG. 6, one gram of activated carbon was sufficient to remove 225 mg folic acid.

### Example 21

### Capture of a Desired MAb from a BZC Clarified Cell Culture Media Using Folic Acid

In a representative experiment described herein, folic acid was used to precipitate a MAb from a representative BZC clarified cell culture media. Accordingly, BZC was used for clarification and folic acid was used for capture.

Folic acid from Example 9 was added to 30 ml of clarified feed from Example 14 (1.7 g/L MAb). The pH of the solution was adjusted to 5.2 using 3M acetic acid and continuously mixed at room temperature for 10 min. After acid addition, a precipitate formed instantly as a result of folic acid complexing with MAb. The precipitate was then collected by centrifugation (4000 rpm for 1 min) and washed with Arginine buffer (0.1M, pH 5.0) to remove loosely-bound impurities. Re-solubilization of the precipitate and elution of MAb took place in 3.5 ml volume at pH 6.75 using 0.1M Thiamine hydrochloride while mixing continuously for 10 min at room temperature. Removal of the free folic acid was effected by adding 0.15 g of activated carbon (NUCHER SA-20, Meadwestvaco, Covington, Va.) to 2 ml of the elution with continuous mixing at room temperature for 10 min. The activated carbon was then collected by centrifugation (4000 rpm for 2 min) and the supernatant filtered through 5 and 0.2μ Millex® filters available from Millipore Corporation of Billerica, Mass. The purified IgG molecule is then recovered in the supernatant fluid.

Under these conditions, >95% of the IgG present in the original fluid bound to folic acid, 88% of IgG was recovered upon elution and 99.8% of folic acid was removed.

### Example 22

### Measurement of HCP Levels in a BZC Clarified Solution Containing a Desired Biomolecule (MAb) that was Captured Using Folic Acid

The experiment was carried out as illustrated in Example 21. An ELISA assay kit (CYGNUS #F550) was used to track the level of host cell protein (HCP) at different steps of the product (MAb) capture process.

The concentration of HCP was reduced from 44247 ng/ml in the starting clarified cell culture fluid to 6500 ng/ml in the elution sample after the folic acid removal step, thereby demonstrating a reduction in HCP levels by 85%. The reported level of HCP in the elution takes into consideration that the starting feed volume was 30 ml but elution volume was 3.5 ml.

### Example 23

### Estimation of Amount of Nitro Red Required to Precipitate MAb

In a representative experiment, another small molecule (i.e., Nitro red which is a dye) was evaluated for precipitation of a MAb molecule. The mass ratio of MAb to Nitro red necessary to precipitate the MAb with more than 90% efficiency was determined.

Feed from Example 5 was titrated to pH 4.5 using 3 M acetic acid. 5 ml aliquot of this solution was then mixed at room temperature for 5 minutes with different volumes of Nitro red from Example 11 to obtain the desired Nitro red to MAb ratio in the solution. The Nitro red to MAb ratio studied in this Example were 0, 0.2, 0.4, 0.8, 1.2, 1.6, 2.0, and 3.0. The mixture was later centrifuged at 3000 rpm for 1 min. The supernatant was removed by decanting, and analyzed for IgG using Protein A HPLC.

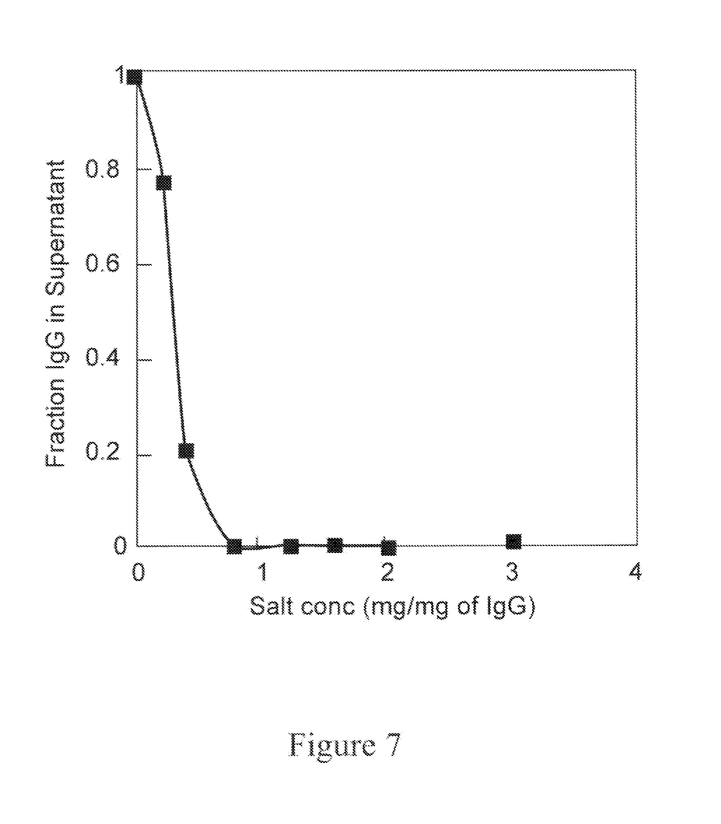

As depicted in FIG. 7, a Nitro red/MAb ratio of 0.8 is required for complete precipitation of MAb.

### Example 24

### Dependence of Binding pH on Elution Recovery of Nitro Red-Precipitated MAb

In a representative experiment, the effect of binding pH on MAb recovery following elution was evaluated.

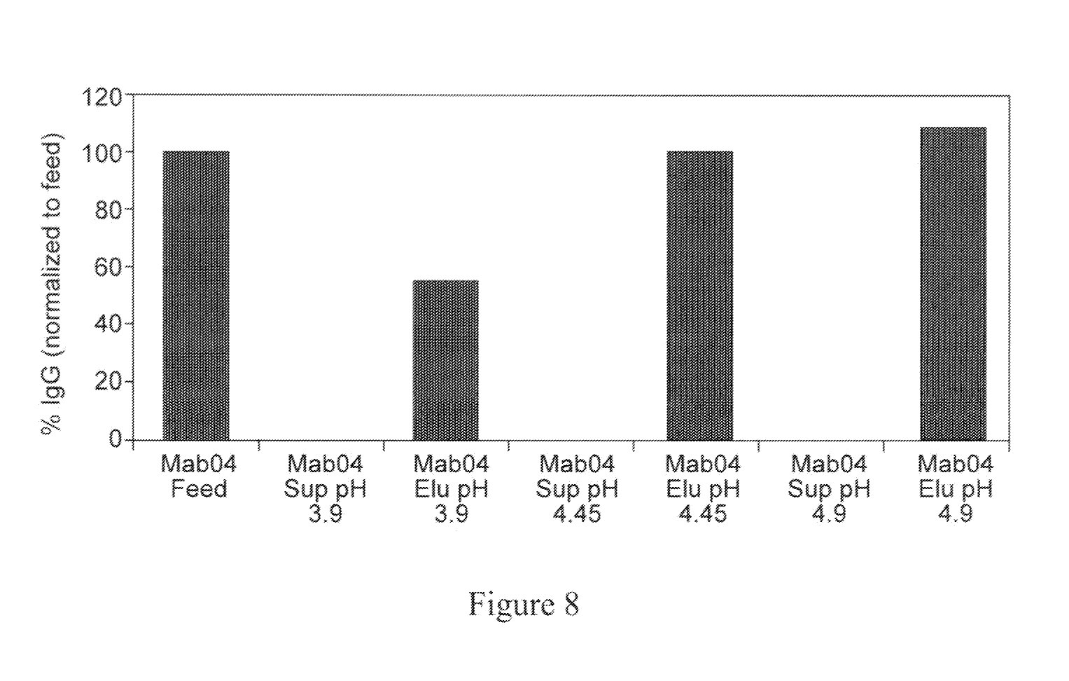

MAb-spiked CCF from Example 5 was titrated to either pH 3.9, 4.5, or 4.9 using 3 M acetic acid. 5 ml aliquot of each of the pH solutions was then mixed at room temperature for 5 minutes with Nitro red from Example 11 to obtain the desired Nitro red to MAb ratio of 1:1. The mixture was centrifuged at 3000 rpm for 1 min. The supernatant was removed by decanting, and passed through Chromasorb (MILLIPORE) to remove residual Nitro red. The solution was then analyzed for MAb using Protein A HPLC. In all 3 cases, there was no MAb left in the supernatant, as depicted in FIG. 8. The precipitates from the 3 different binding pHs were eluted in 20 mM HEPES, pH 8.0+150 mM NaCl. The elution was passed through Chromasorb (MILLIPORE) to remove residual Nitro red, and analyzed for MAb using Protein A HPLC.

As depicted in FIG. 8, lower binding pH (pH 3.9) gave 55% elution recovery, whereas binding at pH 4.5 and 4.9 gave ˜100% yield.

### Example 25

### MAb Recovery and HCP Clearance in Harvested Cell Culture Treated with Amaranth Dye

In this representative experiment, yet another small molecule which is a dye (Amaranth dye) was used to precipitate MAb from a representative clarified cell culture media.

MAb-spiked feed CCF from Example 5 was titrated to pH 4.5 using 3 M acetic acid. The MAb concentration in the MAb-spike feed was 0.95 mg/ml as measured by Protein A HPLC. The host cell protein concentration was 186,000 ng/ml as measured using ELISA (CYGNUS #F550). 5 ml of the solution was mixed with 75 μl of 40 mg/ml Amaranth dye from Example 10 at room temperature for 5 minutes to form a precipitate. The mixture was centrifuged at 3000 rpm for 1 min. The supernatant was removed by decanting, and discarded. The precipitate was redissolved/eluted in 20 mM HEPES, pH 8.0+150 mM NaCl. The elution was treated with 4 mg of activated carbon per ml of eluant to remove any residual Amaranth, and analyzed for MAb recovery using Protein A HPLC and HCP level using ELISA.

A MAb recovery of 96% was obtained and the final HCP levels were 87,100 ng/ml, thereby demonstrating a ˜50% decrease in HCP levels.

### Example 26

### Analysis of Charge Variants of MAb after Precipitation Using Amaranth Dye

In a representative experiment described herein, after precipitating the MAb with Amaranth dye, washing the precipitate to remove impurities and eluting the MAb, the population of charged variants of MAb in sample were analyzed using weak cation exchange chromatography and compared with the population of charged MAb variants in the starting feed. The goal of this experiment was to determine whether soluble complexes of Amaranth dye and MAb existed with the recovered MAb, which would be largely undesirable.

The elution from Example 25 was analyzed for MAb charge variants using analytical weak cation exchange column (WCX-10; Dionex Corp.). The buffers used in the run were 10 mM sodium phosphate, pH 6.0 (Buffer A) and 10 mM sodium phosphate, pH 6.0+500 mM NaCl (Buffer B). The following gradient elution profile was used: time=0, 10% Buffer B; time=40 min, 30% buffer B; time=45 min, 95% buffer B; time=46 min, 100% buffer B.

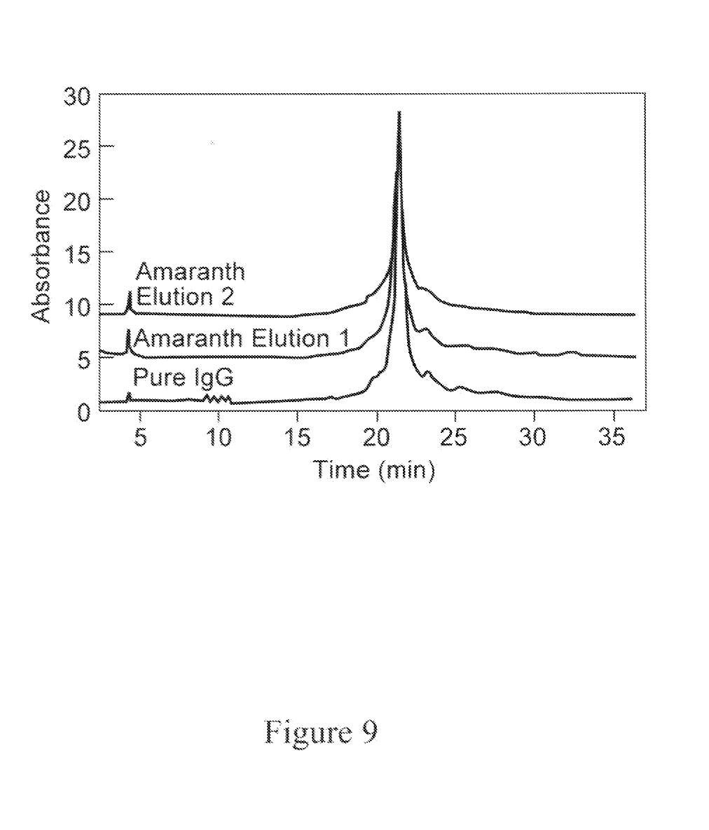

As shown in FIG. 9, no noticeable change in charged variants was observed for the Protein A purified MAb and the Amaranth purified MAb.

### Example 27

### Particle Size Distribution and Effect of Shear on MAb Precipitates Formed Using Folic Acid

In addition to the use of small molecules, such as those described above, which result in adequate purification and MAb recovery with little to no impact on product quality, a precipitation based process also requires steps for handling the precipitate that is formed. A practical technology based on Hollow Fiber Tangential Flow Filtration, TFF, operating in batch and continuous modes, is described herein, which enables efficient handling of the precipitate following the use of small molecules, as described herein.

One of the suitable technologies or steps that may be used for efficient handling of precipitate is a filtration based technology, which depends on the characteristics of the solids that are being processed such as compressibility, particle size, and shear sensitivity, to name a few. For example, if a certain pore size membrane is chosen for the process based on particle size measurements, it is important to confirm that the particle size is not going to change under the influence of the shear rate in the system (for example due to pumping or other mechanical stresses). On the other hand, a particle size smaller than expected may plug the membrane.

As described below, the effect of shear rate on particle size distribution at different binding pH was evaluated in the context of a Hollow Fiber Tangential Flow Filter device.

Feed (30 ml) from Example 2 (0.85 g/L) was spilt into 3 equal parts and mixed for 5 min with folic acid from Example 9 at room temperature. The ratio of folic acid to MAb added was 1:1 for 2 of the aliquots (for titration to pH 4.0 and 5.0), and 1.5:1 for 1 of the aliquot (for later titration to pH 5.5). The 3 aliquots of 10 ml each of the folic acid-mixed feed were titrated to either pH 4.0, 5.0 or 5.5 using 3 M acetic acid. The precipitate was ˜10× diluted (or to a dilution to get enough signal on the instrument) in the appropriate buffer for reading on the Malvern mastersizer to determine the particle size distribution. For precipitate at pH 4.0, 20 mM sodium acetate, pH 4.0 was used. For precipitate at pH 5.0, 20 mM sodium acetate, pH 5.0 was used. For precipitate at pH 5.5, 20 mM sodium acetate, pH 5.5 was used. In addition, the diluted precipitate was passed through a hollow fiber device (0.2 um Midget hoop, GE HEALTHCARE) before entering the measurement chamber in the Malvern instrument. This was done to study the effect of shear on the particle size distribution of the precipitates generated. The flow rate through the hollow fiber was varied in order to generate different degrees of shear. A 5 min equilibration time was given before any measurements.

It was also observed that the total percentage of precipitate (also referred to herein as the solid phase) at the lower pH was generally lower (pH 4.0-11% solids, pH 5.0-14% solids, and pH 5.5-16% solids). The percent solid were calculated based on a centrifuge spin of 3000 rpm for 1 min in a swing-bucket centrifuge. Shear rate (Y) in a pipe for a Newtonian fluid can be measure using the expression: Y=4Q/πr³, where Q is the volumetric flow rate and r is the radius of the pipe.

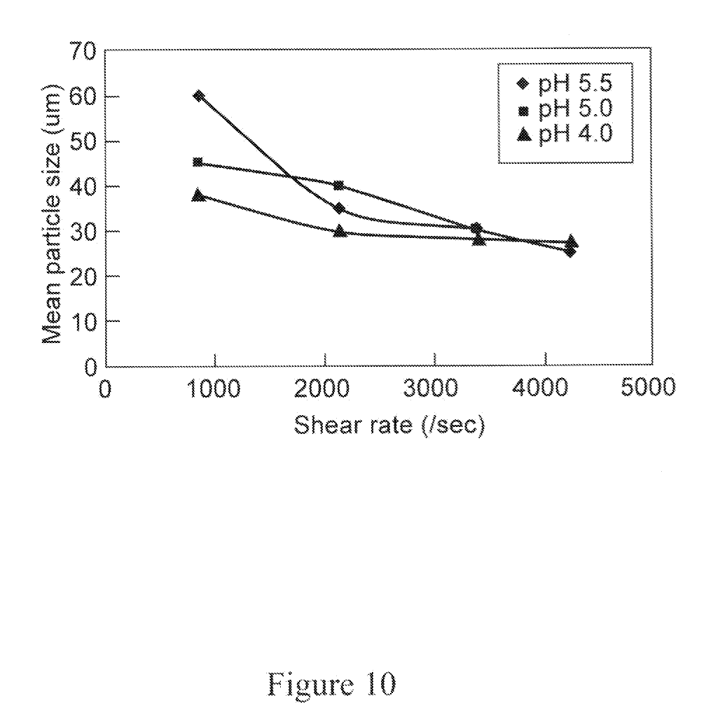

FIG. 10 illustrates the impact of shear on the mean particle size at the different pH conditions tested. Particle size decreases as shear rate increases. It is interesting to note that the particles are more compact and more resistant to shear at the lower binding pH. For the subsequent experiment, a binding pH of 4.5 was chosen.

### Example 28

### Measurement of Flux Versus Transmembrane Pressure (TMP) at Different Shear Rates Using a 0.2 um Hollow Fiber Membrane for MAb Precipitates Generated Using Folic Acid

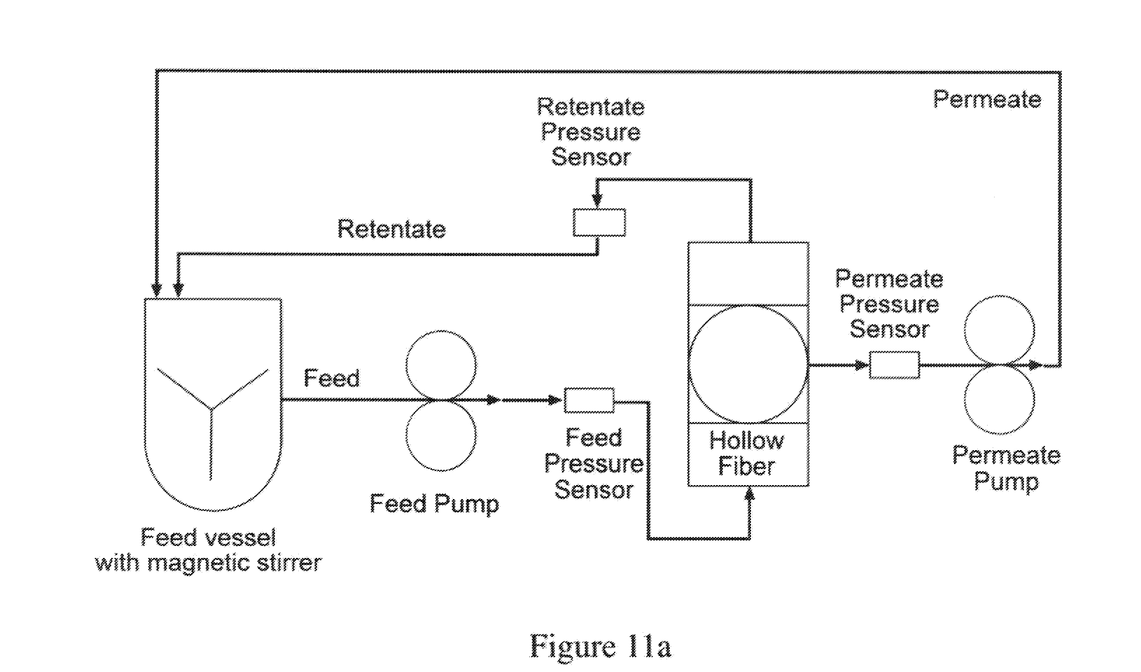

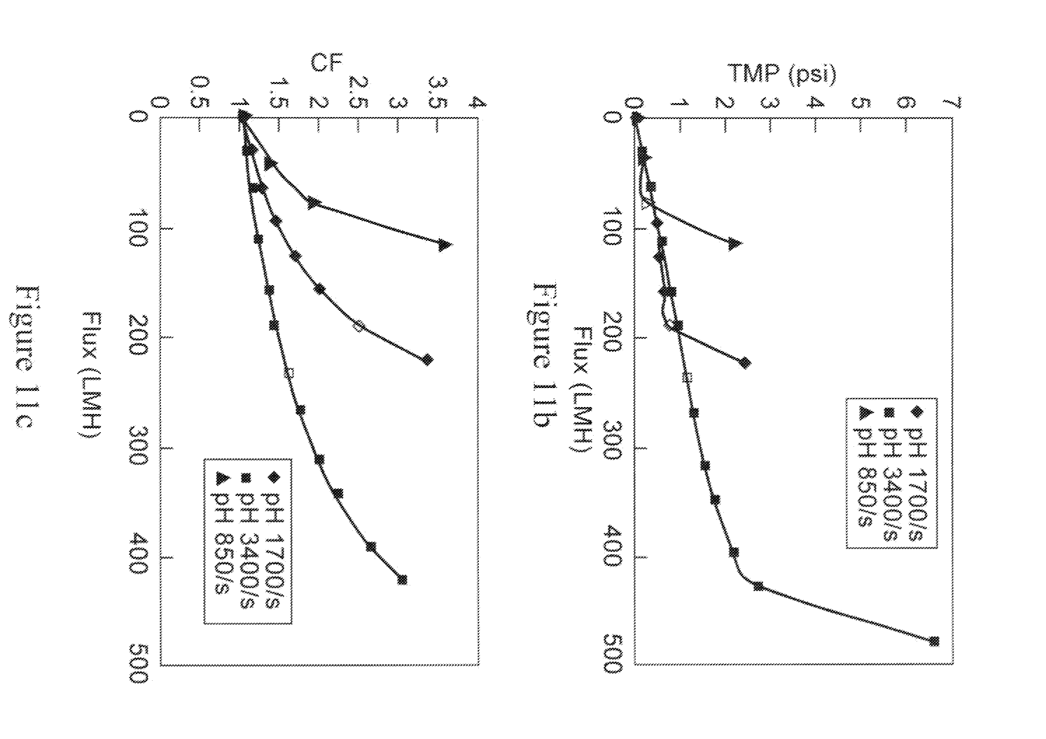

This representative experiment was carried out to determine the optimal shear rate and flux required for stable operation of hollow fiber tangential follow filtration. TFF, system. The latter was set-up under complete recycle mode as shown in FIG. 11a.

Feed (200 ml) from Example 2 (at 0.85 g/L) was mixed for 5 min with folic acid from Example 9 at room temperature such that the ratio of folic acid to MAb was 1:1. The pH of the mixture was then lowered to pH 4.5. For a given feed flow rate (shear rate), the permeate flow rate (permeate flux) was gradually increased in step increments. The feed pressure, retentate pressure, and permeate pressure was monitored for 5 min. The transmembrane pressure was calculated using TMP=(P_{f} Pᵣ)/2−Pₚ. The system was considered at steady state if no change in TMP was observed over 5 min. The membrane used in this study was a 0.2 μm hollow fiber membrane with 38 cm² membrane area (GE HEALTHCARE). The flux vs. TMP is shown in FIG. 11b for 3 different feed flow rates (shear rates). The concentration factor for a single pass (defined as CF=1/(1-Qp/Qf)) as a function of flux is also shown (FIG. 11c). Qp is the permeate flow rate and Qf is the feed flow rate.

It could be inferred from the Flux vs. TMP curves in FIG. 11b that the optimal shear and flow rates rate are 1700 S⁻¹ and 190 LMH, respectively. As depicted in FIG. 11c, under these conditions the maximum concentration factor for a single pass is 2.5×.

### Example 29

### Measurement of Flux Versus Transmembrane Pressure (TMP) at Different Shear Rates Using a 0.2 um Hollow Fiber Membrane for MAb Precipitates Generated Using Folic Acid from a Cell Culture Feed with 4.3 g/L IgG Concentration

For feeds with higher MAb titer, more precipitant (for example folic acid) must be used. Thus a higher starting solid volume needs to be processed. The following experiment was carried out to determine the effect of higher solid content on the performance of the TFF system described in Example 28.

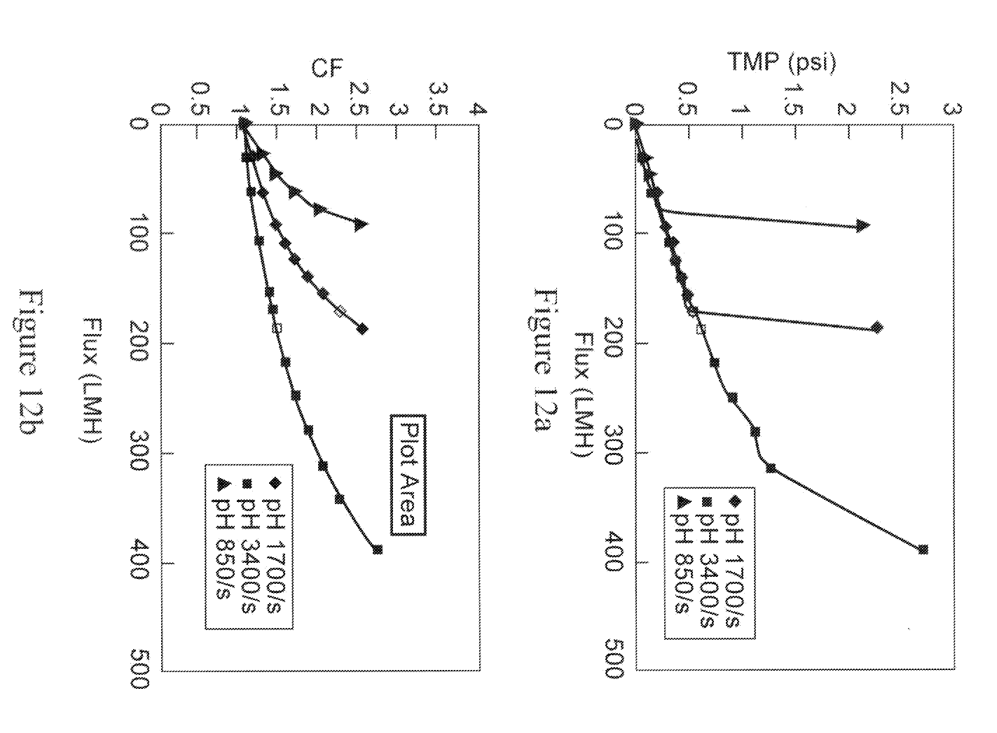

Feed (200 ml) from Example 2 was spiked with pure MAb to obtain a MAb concentration of 4.3 g/L. The MAb-spiked feed was mixed for 5 min with folic acid from Example 9 at room temperature such that the ratio of folic acid to MAb was 1:1. The pH of the mixture was then lowered to pH 4.5. The system was set-up under complete recycle mode as shown in FIG. 11a. For a given feed flow rate (shear rate), the permeate flow rate (permeate flux) was gradually increased in step increments. The feed pressure, retentate pressure, and permeate pressure was monitored for 5 min. The transmembrane pressure was calculated using TMP=(P_{f}+Pᵣ)/2−Pₚ. The system was considered at steady state if no change in TMP was observed over 5 min. The membrane used in this study was a 0.2 μm hollow fiber membrane with 38 cm² membrane area (GE HEALTHCARE). The flux vs. TMP is shown in FIG. 12a for 3 different feed flow rates (shear rates). The concentration factor (defined as CF=1/(1−Qp/Qt)) as a function of flux is also shown (FIG. 12b). Qp is the permeate flow rate and Qf is the feed flow rate.

It could be inferred from the Flux vs. TMP curves in FIG. 12a that the optimal shear and flow rates rate are 1700 S⁻¹ and 174 LMH, respectively. As depicted in FIG. 12b, under these conditions the maximum concentration factor for a single pass is 2.2×. This is very close to the operating conditions identified in Example 28 in case of a MAb titer of 1 g/L, suggesting that the system can handle variations in MAb titer, as it relates to solid volumes.

### Example 30

### MAb Recovery in Harvested Cell Culture Treated with Folic Acid and Processed Using the TFF System Described in Example 28

Feed (250 ml) from Example 2 (1.8 g/L) was mixed for 5 min with folic acid from Example 9 at room temperature such that the ratio of folic acid to IgG was 1:1. The pH of the mixture was then lowered to pH 5.0. The precipitate had about 11% solids. The system was set-up similar to the system illustrated in FIG. 11a (Example 28), except that the permeate line was not re-cycled to feed but sent to a separate collection beaker for IgG quantification. The precipitate was concentrated ˜4.0× to a final volume of 63 ml at constant transmembrane pressure (the TMP was maintained between 0.4-0.5 psi) by controlling the permeate flux. The average flux during the concentration phase was 75 LMH. Following concentration, the solids were washed with 120 ml of 0.1 M Arginine, pH 5.0. Washing was accomplished by pumping wash buffer into the feed beaker at the same flow rate as the permeate flow rate (70 LMH). The permeate from the wash was also collected for MAb quantification. The solids were then redissolved eluted by increasing the pH to 7.0 using 2 M Tris-base (pH 10) and addition of Thiamine to achieve a final Thiamine concentration of 0.1 M. No MAb was observed in the permeate either during concentration or wash. The overall MAb recovery was 87%, and a ˜3.0X concentration could be achieved.

### Example 31

### Kinetics of Precipitate Formation in a Static Mixer

In addition to operating the TFF system in batch mode, feasibility of the continuous mode operation was evaluated. One pre-requisite for continuous operation is fast binding and precipitation kinetics so that an inline mixer can be used to continuously feed the TFF system. The following representative experiment describes the kinetics of precipitate formation using a static mixer.

Feed (50 ml) from Example 2 (0.85 g/L) was mixed for 5 min with folic acid from Example 9 at room temperature such that the ratio of folic acid to MAb was 1:1. This solution was then pumped at 10 ml/min through a helical static mixer (Cole Palmer) with a dead volume of <5 ml. A 3M acetic acid stream at 0.26 ml/min was introduced prior to the static mixer using a T-joint. The residence time in the static mixer was <30 sec. Five fractions with 10 ml volume each were collected and the pH was measured and confirmed to be around 4.5. This indicated that the static mixer allows for steady state operation and that the pH could be consistently maintained at the desired level. The samples were then centrifuged at 2500 rpm for 1 min. The supernatant was then analyzed for MAb concentration using Protein A HPLC.

No MAb was observed in the supernatant indicating complete precipitation of MAb occurred within 30 sec.

### Example 32

### Concentration and Washing of Solids Using a Hollow Fiber TFF in Continuous Countercurrent Mode

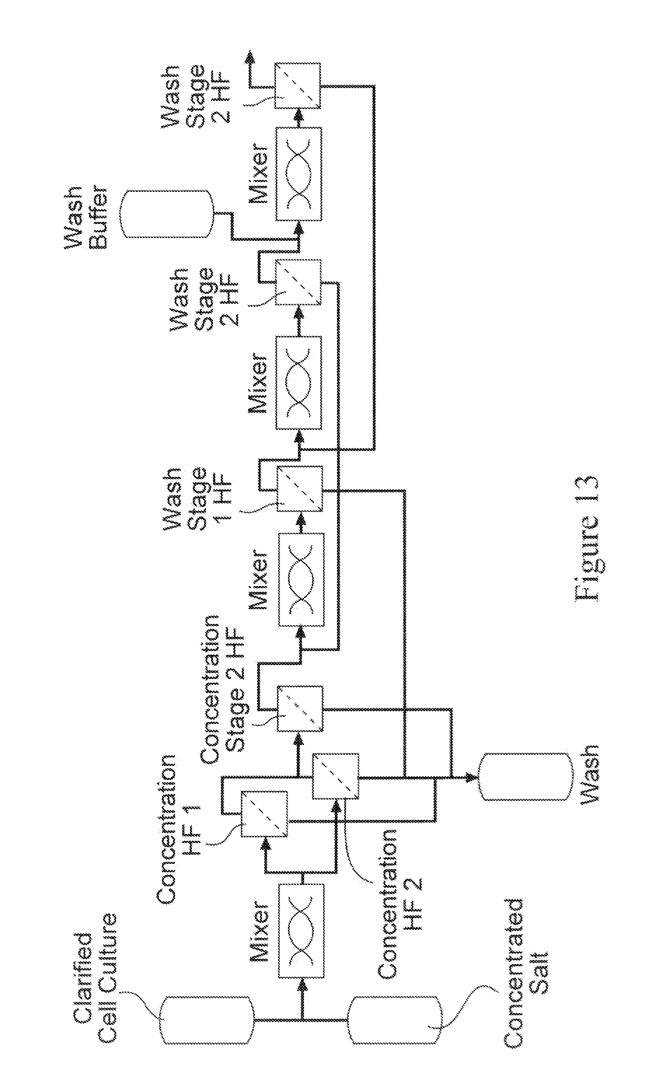

A hollow fiber tangential flow filtration system was set up to operate in continuous mode as described in FIG. 13. The following experiment describes the processing conditions used and the resulting MAb recovery.

Feed (2000 ml) from Example 2 (1.8 g/L) was mixed for 5 min with folic acid from Example 9 at room temperature such that the ratio of folic acid to MAb was 1:1. The pH of the mixture was then lowered to pH 5.0. The precipitate had about 11% solids. The precipitate was concentrated 4×, in two steps, to a final volume of 500 ml at 197 LMH permeate flux. Following concentration, the solids were washed with 314 ml of 25 mM sodium acetate, pH 5. Washing was performed in a countercurrent setup, fresh wash buffer was pumped into feed entering final hollow fiber device and the permeate from the final device was used as the wash buffer for the previous device and that permeate was used as the wash buffer for first device. The solids were then redissolved/eluted by increasing the pH to 7.0 using 2 M Tris-base (pH 10) followed by addition of Thiamine to a final Thiamine concentration of 0.1 M. The overall MAb recovery was 74%. There was no MAb loss in the permeate in either of the concentration or wash steps.

### Example 33

### Complete MAb Downstream Purification Process, which Employs a Clarification Step with BZC, a Capture Step with Folic Acid, and One or More Polishing Steps for Increased Purity with Activated Carbon and Anion-Exchange Membrane Chromatography

The goal of this experiment was to demonstrate that the entire downstream purification of a monoclonal antibody can be achieved using precipitation in the clarification and capture steps followed by flow through purification steps

Feed from example 21 was diluted 4-fold with aqueous Tris buffer solution, 25 mM, pH 7.0, and the final pH was adjusted to 7.0. Powdered activated carbon was obtained from MeadWestVaco Corporation, Richmond, Va., USA as Nuchar HD grade. Glass Omnifit Chromatography Column (10 mm diameter, 100 mm length) was loaded with 250 mg of HD Nuchar activated carbon slurried in Water to give a packed column volume of 1 mL. The column was equilibrated with aqueous Tris buffer solution, 25 mM, pH 7.0, 0.2 mL ChromaSorb membrane devices were manufactured using 0.65 micron-rated polyethylene membrane modified with polyallyl amine, available from Millipore Corporation, Billerica, Mass., USA, in devices of various sizes. The membrane was cut in 25 mm discs; 5 discs were stacked and sealed in an overmolded polypropylene device of the same type as the OptiScale 25 disposable capsule filter devices commercially available from Millipore Corporation. The devices include an air vent to prevent air locking, and have an effective filtration area of 3.5 cm² and volume of 0.2 mL.

The diluted monoclonal antibody feed was pumped through the activated carbon column at a constant flow rate of 0.1 ml/min, to obtain the flow-through pool of 200 ml (200 column volumes). A portion of this pool was flowed through a 0.2 mL ChromaSorb device to obtain a flow-through pool of 8 ml (40 column volumes). The purity of the samples is listed in Table 2.

The final purity of the antibody was at about 14 ppm of HCP, indicates that the template described herein, is a feasible and competitive downstream purification process that achieves acceptable purification and mab recovery targets.

TABLE 2

| | | MAb | |
|---|---|---|---|
| | | concentration | Cumulative |
| Sample | HCP (ppm) | (g/L) | MAb yield (%) |
| Folic acid elution | 4508 | 4.38 | 100 |
| (Example Y) | | | |
| Activated carbon | 401 | 4.00 | 91 |
| flow-through | | | |
| ChromaSorb flow- | 14 | 3.57 | 81 |
| through | | | |

### Example 34

### Clarification of Cell Culture Media and Subsequent Clearance of HCP Using HTAB

Feed (200 ml) from Example 3 was spiked with pure MAb to obtain a MAb concentration of 4.8 g/L. The HCP concentration in the feed was about 179,000 ng/ml. 2 ml of HTAB from Example 7 was added to 38 ml of the above feed and mixed at room temperature for 10 minutes, in order to allow for binding and precipitation of insoluble impurities, such as cells and cell debris as well as soluble impurities, such as host cell proteins, nucleic acids, etc. The precipitate was then collected by centrifugation (4000 rpm for 1 min) and the supernatant filtered through 0.2μ Durapore® filter. Under these conditions, 100% of the MAb present in the original fluid was recovered and 95% of the HCP was removed.

The specification is most thoroughly understood in light of the teachings of the references cited within the specification which are hereby incorporated by reference. The embodiments within the specification provide an illustration of embodiments in this invention and should not be construed to limit its scope. The skilled artisan readily recognizes that many other embodiments are encompassed by this invention. All publications and inventions are incorporated by reference in their entirety. To the extent that the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

What is claimed is:

1. A method of separating a target biomolecule from one or more insoluble impurities in a sample: the method comprising the steps of:
(i) providing a sample comprising a biomolecule of interest and one or more insoluble impurities;
(ii) contacting the sample with a small molecule comprising at least one cationic group and at least one non-polar group, in an amount sufficient to form a precipitate comprising the one or more insoluble impurities; and
(iii) removing the precipitate from the sample, thereby to separate the target molecule from the one or more insoluble impurities.

2. The method of claim 1, where the non-polar group is aromatic.

3. The method of claim 1, wherein the non-polar group is aliphatic.

4. The method of claim 1, wherein the one or more insoluble impurities are selected from whole cells and cell debris.

5. The method of claim 1, wherein the small molecule is selected from the group consisting of a monoalkyltrimethyl ammonium salt, a monoalkyldimethylbenzyl ammonium salt, a dialkyldimethyl ammonium salt, a heteroaromatic ammonium salt, a polysubstituted quaternary ammonium salt and a bis-quaternary ammonium salt.

6. The method of claim 5, wherein a monoalkyltrimethyl ammonium salt is selected from the group consisting of cetyltrimethylammonium bromide, cetyltrimethylammonium chloride, tetradecyltrimethylammonium bromide, tetradecyltrimethylammonium chloride, alkyltrimethyl ammonium chloride, alkylaryltrimethyl ammonium chloride, dodecyltrimethylammonium bromide, dodecyltrimethylammonium chloride, dodecyldimethyl-2-phenoxyethylammonium bromide, hexadecylamine chloride, hexadecylamine bromide, dodecyl amine, dodecyl chloride, cetyldimethylethyl ammonium bromide and cetyldimethylethyl ammonium chloride.

7. The method of claim 6, wherein a monoalkyldimethylbenzylammonium salt is selected from the group consisting of alkyldimethylbenzyl ammonium chloride and benzethonium chloride.

8. The method of claim 6, wherein a dialkyldimethyl ammonium salt is selected from the group consisting of domiphen bromide, didecyldimethyl ammonium chloride, didecyldimethyl ammonium bromide, octyldodecyldimethyl ammonium chloride and octyldodecyldimethyl ammonium bromide.

9. The method of claim 6, wherein a heteroaromatic ammonium salt is selected from the group consisting of cetylpyridium chloride, cetylpyridium bromide, hexadecylpyridinium bromide, hexadecylpyridinium chloride, cis-isomer 1-[3-chloroallyl]-3,5,7-triaza-1-azoniaadamantane, alkyl-isoquinolinium bromide and alkyldimethylnaphthylmethyl ammonium chloride.

10. The method of claim 6, wherein a polysubstituted quaternary ammonium salt is selected from the group consisting of alkyldimethylbenzyl ammonium saccharinate and alkyldimethylethylbenzyl ammonium cyclohexylsulfamate.

11. The method of claim 1, wherein the small molecule is benzethonium chloride

12. The method of claim 1, wherein the amount of small molecule added in step (ii) ranges 0.01 to 2.0% wt/vol.

13. The method of claim 1, wherein the small molecule is added in solution form in step (ii) at a concentration ranging from 1 to 200 mg/ml.

14. The method of claim 1, wherein the precipitation of one or more insoluble impurities is carried out at a pH ranging from 2 to 9.

15. The method of claim 1, wherein the removal of the precipitate in step (iii) comprises use of filtration.

16. The method of claim 1, wherein the removal of precipitate in step (iii) comprises use of centrifugation.

17. The method of claim 1, further comprising the step of removing residual amounts of small molecule from the sample containing the target biomolecule after removal of the precipitate.

18. The method of claim 17, wherein the step of removing residual amounts of small molecule comprises contacting the sample with a polyanion.

19. The method of claim 17, wherein the step of removing residual amounts of small molecule comprises contacting the sample with an adsorbant material.

20. The method of claim 17, wherein the step of removing residual amounts of small molecule comprises contacting the sample with activated carbon.

21. A method of purifying a target biomolecule from a sample comprising the target molecule along with one or more soluble impurities, wherein the method comprises the steps of:
(i) contacting the sample with a small molecule comprising at least one anionic group and at least one non-polar group, in an amount sufficient to form a precipitate comprising the target molecule; and
(ii) recovering the precipitate, thereby to separate the target biomolecule from the one or more soluble impurities.

22. The method of claim 21, wherein the non-polar group is aromatic.

23. The method of claim 21, wherein the non-polar group is aliphatic.

24. The method of claim 21, wherein the sample is subjected to a clarification step prior to step (i).

25. The method of claim 24, wherein the clarification step comprises use of filtration.

26. The method of claim 24, wherein the clarification step comprises use of centrifugation.

27. The method of claim 24, wherein the clarification step comprises contacting the sample with a small molecule comprising at least one cationic group and at least one non-polar group.

28. The method of claim 21, wherein the small molecule is selected from the group consisting of a pterin derivative, etacrynic acid, fenofibric acid, mefenamic acid, mycophenolic acid, tranexamic acid, zoledronic acid, zcetylsalicylic acid, arsanilic acid, ceftiofur acid, meclofenamic Acid, ibuprofine, naproxen, fusidic acid, nalidixic acid, chenodeoxycholic acid, ursodeoxycholic acid, tiaprofenic acid, niflumic acid, trans-2-hydroxycinnamic acid, 3-phenylpropionic acid, probenecid, clorazepate, icosapent, 4-acetamidobenzoic acid, ketoprofen, tretinoin, adenylosuccinic acid, naphthalene-2,6-disulfonic acid, tamibarotene, etodolacetodolic acid and benzylpenicillinic acid.

29. The method of claim 28, wherein the pterin derivative is selected from folic acid and pteroic acid.

30. The method of claim 21, wherein the small molecule is folic acid or a derivative thereof.

31. The method of claim 21, wherein the small molecule is a dye molecule.

32. The method of claim 31, wherein the dye molecule is Amaranth or Nitro red.

33. The method of claim 21, wherein the small molecule is added to a concentration ranging from 0.001% to 5.0%.

34. The method of claim 21, wherein the pH of sample is adjusted prior to the addition of the small molecule.

35. The method of claim 21, wherein the precipitation is carried out at a pH ranging from 2 to 9.

36. The method of claim 21, wherein the amount of target biomolecule present in the precipitate is at least 50%, or at least 60%, or at least 70%, or at least 80%, or at least 90% or greater than 90% of the initial target biomolecule amount in the sample.

37. The method of claim 21, wherein the impurity level in the precipitate is less than 50%, or less than 40%, or less than 30%, or less than 20%, or less than 15%, or less than 10%, or less than 5% of the initial impurity level present in the sample.

38. The method of claim 21, further comprising the step of dissolving the precipitate comprising the target biomolecule in a suitable buffer.

39. The method of claim 38, wherein the buffer comprises a pH ranging from 4.5 to 10.

40. The method of claim 21, further comprising one or more chromatography steps.

41. The method of claim 40, wherein the one or more chromatography steps are selected from the group consisting of ion exchange chromatography, hydrophobic interaction chromatography, affinity chromatography, and mixed mode chromatography.

42. The method of claim 1, wherein the target biomolecule is selected from the group consisting of a recombinant protein, an antibody or a functional fragment thereof, a CH2/CH3 region-containing protein and an immunoadhesion molecule.

43. The method of claim 21, wherein the target biomolecule is selected from the group consisting of a recombinant protein, an antibody or a functional fragment thereof, a CH2/CH3 region-containing protein and an immunoadhesion molecule.

44. The method of claim 42, wherein the antibody is selected from a monoclonal antibody, a polyclonal antibody, a humanized antibody, a chimeric antibody and a multispecific antibody.

45. The method of claim 1, wherein the target biomolecule is produced by expression in a mammalian cell.

46. The method of claim 1, wherein the target biomolecule is produced by expression in a non-mammalian cell.

47. The method of claim 21, wherein the target biomolecule is produced by expression in a mammalian cell.

48. The method of claim 21, wherein the target biomolecule is produced by expression in a non-mammalian cell.

49. A method of purifying an antibody in a sample, the method comprising the steps of:
(i) providing a sample comprising an antibody and one or more insoluble impurities;
(ii) contacting the sample with a small molecule comprising at least one cationic group and at least one non-polar group, in an amount sufficient to form a precipitate comprising the one or more insoluble impurities and a liquid phase comprising the antibody; and
(iii) subjecting the liquid phase to at least one chromatography step, thereby to purify the antibody.

50. The method of claim 49, wherein the small molecule is added to the sample using one or more static mixers.

51. The method of claim 49, wherein the at least one chromatography step is an affinity chromatography step.
